(19) United States
(12) Patent Application Publication
(10) Pub. No.: US 2020/0319213 A1
(43) Pub. Date: Oct. 8, 2020

(54) DETECTION OF HORMONES IN HAIR SAMPLES AND OTHER BIOLOGICAL SAMPLES

(71) Applicant: MedAnswers, Inc., San Pedro, CA (US)

(72) Inventors: Manel LOPEZ BEJAR, Montclair, CA (US); Santiago MUNNE, Short Hills, NJ (US); Alice CRISCI, San Pedro, CA (US); Sarthak SAWARKAR, Lawrence Township, NJ (US)

(21) Appl. No.: 16/897,576

(22) Filed: Jun. 10, 2020

Related U.S. Application Data
(63) Continuation of application No. PCT/US2019/064556, filed on Dec. 4, 2019.
(60) Provisional application No. 62/911,105, filed on Oct. 4, 2019.

(30) Foreign Application Priority Data
Dec. 5, 2018 (EP) 18382898.7

(51) Int. Cl. G01N 33/74; G01N 33/68

(52) U.S. Cl. CPC G01N 33/743; G01N 33/6893

(57) ABSTRACT

The present disclosure provides methods and systems for generating a reproductive hormone profile of a subject. A method for generating a reproductive hormone profile of a subject may comprise (a) obtaining a hair sample of the subject; (b) processing the hair sample to generate data indicative of a presence of a reproductive hormone in the hair sample; and (c) processing at least some of the data of (b) using a reproductive hormone classifier to generate the reproductive hormone of the subject. The present disclosure also provides compositions and kits for performing methods described herein or using the systems described herein.

### CROSS-REFERENCE

This application is a continuation of PCT Application No. PCT/US2019/064556, filed Dec. 4, 2019, which claims the benefit of European Patent Application No. EP18382898.7, filed Dec. 5, 2018, and U.S. Provisional Patent Application No. 62/911,105, filed Oct. 4, 2019, each of which is entirely incorporated herein by reference.

### BACKGROUND

Assays of hormones such as dehydroepiandrosterone (DHEA), estradiol, progesterone, testosterone, cortisol, prolactin, vitamin D, and anti-mullerian hormone (AMH) may be performed using plasma or serum. However, such assays may not be able to accurately determine long-term retrospective hormone levels.

### SUMMARY

The success rates of conception and assisted reproductive techniques may need improvement. As such, the present disclosure provides compositions, methods, systems, devices, platforms, and kits for detecting and/or quantifying a presence of biologically active forms of hormones. Such detection or quantification may be conducted on a hair sample. Methods of the present disclosure may include reproductive medicine, endocrinology, biochemistry, and related technologies.

In an aspect, the present disclosure provides a method for generating a reproductive hormone profile of a subject, comprising: (a) obtaining a hair sample of the subject; (b) processing the hair sample of the subject to generate data indicative of a presence of a reproductive hormone in the hair sample; and (c) processing at least the data of (b) using a reproductive hormone classifier to generate the reproductive hormone profile of the subject.

In some embodiments, the subject is a human. In some embodiments, the reproductive hormone is selected from the group consisting of dehydroepiandrosterone, estradiol, progesterone, testosterone, anti-mullerian hormone, prolactin, and vitamin D. In some embodiments, processing the hair sample in (b) further comprises measuring a presence of cortisol, wherein the data set comprises additional data indicative of the presence of cortisol, and wherein (c) comprises processing the additional data using the reproductive hormone classifier to generate the reproductive hormone profile of the subject. In some embodiments, processing the hair sample in (b) further comprises measuring a micronutrient, wherein the data set comprises additional data indicative of the presence of the micronutrient, and wherein (c) comprises processing the additional data using the reproductive hormone classifier to generate the reproductive hormone profile of the subject. In some embodiments, the micronutrient is selected from the group consisting of folic acid, vitamin B12, lithium, vitamin B1, vitamin B2, vitamin B3, vitamin B5, vitamin B6, iron, iodine, phosphorus, potassium, selenium, and retinyl ester.

In some embodiments, the reproductive hormone profile is an assessment of ovarian reserve in the subject. In some embodiments, the reproductive hormone profile is an assessment of reproductive lifespan of the subject. In some embodiments, the reproductive hormone profile is an assessment of ovarian dysfunction of the subject. In some embodiments, the ovarian dysfunction is selected from the group consisting of polycystic ovary syndrome, endometriosis, anovulation, persistent follicles, and granulosa cell cancer.

In some embodiments, the reproductive hormone profile is an assessment of the subject after the human subject undergoes gonadotoxic cancer treatment. In some embodiments, the reproductive hormone profile is an assessment of the subject after the subject undergoes a complete oophorectomy. In some embodiments, the reproductive hormone profile is an assessment of the subject after the subject undergoes a partial oophorectomy. In some embodiments, the reproductive hormone profile is an assessment of metabolic syndromes, such as obesity, insulin resistance, and type 2 diabetes. In some embodiments, the reproductive hormone profile includes an assessment of vitamin D deficiency in the subject. In some embodiments, the reproductive hormone profile includes an assessment of thyroid dysfunction in the subject.

In some embodiments, the method further comprises obtaining a first hair sample of the subject subsequent to the subject undergoing initiation of an assisted reproductive technique. In some embodiments, the method further comprises obtaining a second hair sample of the subject subsequent to the subject undergoing initiation of an assisted reproductive technique. In some embodiments, the first hair sample or the second hair sample is selected from hair samples collected from the subject's axilla, pubic area, and head.

In some embodiments, (b) further comprises using the hair sample to generate a solution suspected of containing the reproductive hormone and assaying the solution for the presence of the reproductive hormone. In some embodiments, (b) further comprises using the hair sample to generate a solution suspected of containing the micronutrient, and assaying the solution for said presence of said micronutrient

In some embodiments, the hair sample is obtained at a location that is remotely located with respect to a location of the subject. In some embodiments, the hair sample is obtained from the remote location using a delivery service. In some embodiments, the reproductive hormone classifier comprises a trained machine learning algorithm.

In some embodiments, (c) further comprises generating an electronic report having the reproductive hormone profile of the subject. In some embodiments, the electronic report is provided for display on an electronic device of the subject.

In some embodiments, the reproductive hormone profile indicates a deficiency or abundance of a reproductive hormone in the subject. In some embodiments, the method further comprises treating the subject for a reproductive disease or disorder based at least in part on the reproductive hormone profile.

In some embodiments, the reproductive hormone classifier identifies a reproductive hormone in the sample, or a deficiency or an abundance of the reproductive hormone in the sample, at an accuracy of at least about 80%. In some embodiments, the reproductive hormone classifier identifies a reproductive hormone in the sample, or a deficiency or an abundance of the reproductive hormone in the sample, at an accuracy of at least about 90%. In some embodiments, the reproductive hormone classifier identifies a reproductive hormone in the sample, or a deficiency or an abundance of the reproductive hormone in the sample, at an accuracy of at least about 95%. In some embodiments, the reproductive hormone classifier identifies a reproductive hormone in the sample, or a deficiency or an abundance of the reproductive hormone in the sample, at a sensitivity of at least about 80%. In some embodiments, the reproductive hormone classifier identifies a reproductive hormone in the sample, or a deficiency or an abundance of the reproductive hormone in the sample, at a sensitivity of at least about 90%. In some embodiments, the reproductive hormone classifier identifies a reproductive hormone in the sample, or a deficiency or an abundance of the reproductive hormone in the sample, at a sensitivity of at least about 95%. In some embodiments, the reproductive hormone classifier identifies a reproductive hormone in the sample, or a deficiency or an abundance of the reproductive hormone in the sample, at a specificity of at least about 80%. In some embodiments, the reproductive hormone classifier identifies a reproductive hormone in the sample, or a deficiency or an abundance of the reproductive hormone in the sample, at a specificity of at least about 90%. In some embodiments, the reproductive hormone classifier identifies a reproductive hormone in the sample, or a deficiency or an abundance of the reproductive hormone in the sample, at a specificity of at least about 95%. In some embodiments, the reproductive hormone classifier identifies a reproductive hormone in the sample, or a deficiency or an abundance of the reproductive hormone in the sample, at a positive predictive value (PPV) of at least about 80%. In some embodiments, the reproductive hormone classifier identifies a reproductive hormone in the sample, or a deficiency or an abundance of the reproductive hormone in the sample, at a positive predictive value (PPV) of at least about 90%. In some embodiments, the reproductive hormone classifier identifies a reproductive hormone in the sample, or a deficiency or an abundance of the reproductive hormone in the sample, at a positive predictive value (PPV) of at least about 95%. In some embodiments, the reproductive hormone classifier identifies a reproductive hormone in the sample, or a deficiency or an abundance of the reproductive hormone in the sample, at a negative predictive value (NPV) of at least about 80%. In some embodiments, the reproductive hormone classifier identifies a reproductive hormone in the sample, or a deficiency or an abundance of the reproductive hormone in the sample, at a negative predictive value (NPV) of at least about 90%. In some embodiments, the reproductive hormone classifier identifies a reproductive hormone in the sample, or a deficiency or an abundance of the reproductive hormone in the sample, at a negative predictive value (NPV) of at least about 95%. In some embodiments, the reproductive hormone classifier identifies a reproductive hormone in the sample, or a deficiency or an abundance of the reproductive hormone in the sample, with an Area Under the Receiver Operating Characteristic (AUROC) of at least about 0.70. In some embodiments, the reproductive hormone classifier identifies a reproductive hormone in the sample, or a deficiency or an abundance of the reproductive hormone in the sample, with an Area Under the Receiver Operating Characteristic (AUROC) of at least about 0.80. In some embodiments, the reproductive hormone classifier identifies a reproductive hormone in the sample, or a deficiency or an abundance of the reproductive hormone in the sample, with an Area Under the Receiver Operating Characteristic (AUROC) of at least about 0.90. In some embodiments, the reproductive hormone classifier identifies a reproductive hormone in the sample, or a deficiency or an abundance of the reproductive hormone in the sample, with an Area Under the Receiver Operating Characteristic (AUROC) of at least about 0.95.

In another aspect, the present disclosure provides a method for generating a reproductive hormone profile of a subject, comprising: (a) obtaining a hair sample of the subject; (b) processing the hair sample of the subject to generate data indicative of a presence of an anti-mullerian hormone (AMH) in the hair sample; and (c) using at least the data in (b) to generate the reproductive hormone profile of the subject.

In some embodiments, the subject is a human. In some embodiments, the method further comprises measuring a presence of another hormone selected from the group consisting of dehydroepiandrosterone, estradiol, progesterone, testosterone, and cortisol.

In some embodiments, the reproductive hormone profile is an assessment of ovarian reserve in the subject. In some embodiments, the reproductive hormone profile is an assessment of reproductive lifespan of the subject. In some embodiments, the reproductive hormone profile is an assessment of ovarian dysfunction in the subject. In some embodiments, the ovarian dysfunction is selected from the group consisting of polycystic ovary syndrome, endometriosis, anovulation, persistent follicles, and granulosa cell cancer.

In some embodiments, the reproductive hormone profile is an assessment of the subject after the subject undergoes gonadotoxic cancer treatment. In some embodiments, the reproductive hormone profile is an assessment of the subject after the subject undergoes a complete oophorectomy. In some embodiments, the reproductive hormone profile is an assessment of the subject after the subject undergoes a partial oophorectomy.

In some embodiments, the method further comprises obtaining a first hair sample of the subject prior to the subject undergoing initiation of an assisted reproductive technique. In some embodiments, the method further comprises obtaining a second hair sample of the subject subsequent to the subject undergoing initiation of an assisted reproductive technique.

In some embodiments, the first hair sample or the second hair sample is selected from hair samples collected from the subject's axilla, pubic area, and head. In some embodiments, (b) further comprises using the hair sample to generate a solution suspected of containing the AMH and assaying the solution for the presence of the AMH.

In some embodiments, the hair sample is obtained at a location that is remotely located with respect to a location of the subject. In some embodiments, the hair sample is obtained from the remote location using a delivery service.

In some embodiments, (c) further comprises generating an electronic report having the reproductive hormone profile of the subject. In some embodiments, the electronic report is provided for display on an electronic device of the subject. In some embodiments, the reproductive hormone profile indicates a deficiency or abundance of a reproductive hormone in the subject.

In some embodiments, the method further comprises treating the subject for a reproductive disease or disorder based at least in part on the reproductive hormone profile. In some embodiments, (c) comprises using a reproductive hormone classifier to identify a reproductive hormone in the sample, or a deficiency or an abundance of the reproductive hormone in the sample, at an accuracy of at least about 80%. In some embodiments, the reproductive hormone classifier identifies a reproductive hormone in the sample, or a deficiency or an abundance of the reproductive hormone in the sample, at an accuracy of at least about 90%. In some embodiments, the reproductive hormone classifier identifies a reproductive hormone in the sample, or a deficiency or an abundance of the reproductive hormone in the sample, at an accuracy of at least about 95%. In some embodiments, (c) comprises using a reproductive hormone classifier to identify a reproductive hormone in the sample, or a deficiency or an abundance of the reproductive hormone in the sample, at a sensitivity of at least about 80%. In some embodiments, the reproductive hormone classifier identifies a reproductive hormone in the sample, or a deficiency or an abundance of the reproductive hormone in the sample, at a sensitivity of at least about 90%. In some embodiments, the reproductive hormone classifier identifies a reproductive hormone in the sample, or a deficiency or an abundance of the reproductive hormone in the sample, at a sensitivity of at least about 95%. In some embodiments, (c) comprises using a reproductive hormone classifier to identify a reproductive hormone in the sample, or a deficiency or an abundance of the reproductive hormone in the sample, at a specificity of at least about 80%. In some embodiments, the reproductive hormone classifier identifies a reproductive hormone in the sample, or a deficiency or an abundance of the reproductive hormone in the sample, at a specificity of at least about 90%. In some embodiments, (c) comprises using a reproductive hormone classifier to identify a reproductive hormone in the sample, or a deficiency or an abundance of the reproductive hormone in the sample, at a specificity of at least about 95%.

In some embodiments, (c) comprises using a reproductive hormone classifier to identify a reproductive hormone in the sample, or a deficiency or an abundance of the reproductive hormone in the sample, at a positive predictive value (PPV) of at least about 80%. In some embodiments, the reproductive hormone classifier identifies a reproductive hormone in the sample, or a deficiency or an abundance of the reproductive hormone in the sample, at a positive predictive value (PPV) of at least about 90%. In some embodiments, the reproductive hormone classifier identifies a reproductive hormone in the sample, or a deficiency or an abundance of the reproductive hormone in the sample, at a positive predictive value (PPV) of at least about 95%. In some embodiments, (c) comprises using a reproductive hormone classifier to identify a reproductive hormone in the sample, or a deficiency or an abundance of the reproductive hormone in the sample, at a negative predictive value (NPV) of at least about 80%. In some embodiments, the reproductive hormone classifier identifies a reproductive hormone in the sample, or a deficiency or an abundance of the reproductive hormone in the sample, at a negative predictive value (NPV) of at least about 90%. In some embodiments, the reproductive hormone classifier identifies a reproductive hormone in the sample, or a deficiency or an abundance of the reproductive hormone in the sample, at a negative predictive value (NPV) of at least about 95%. In some embodiments, (c) comprises using a reproductive hormone classifier to identify a reproductive hormone in the sample, or a deficiency or an abundance of the reproductive hormone in the sample, with an Area Under the Receiver Operating Characteristic (AUROC) of at least about 0.70. In some embodiments, the reproductive hormone classifier identifies a reproductive hormone in the sample, or a deficiency or an abundance of the reproductive hormone in the sample, with an Area Under the Receiver Operating Characteristic (AUROC) of at least about 0.80. In some embodiments, the reproductive hormone classifier identifies a reproductive hormone in the sample, or a deficiency or an abundance of the reproductive hormone in the sample, with an Area Under the Receiver Operating Characteristic (AUROC) of at least about 0.90. In some embodiments, the reproductive hormone classifier identifies a reproductive hormone in the sample, or a deficiency or an abundance of the reproductive hormone in the sample, with an Area Under the Receiver Operating Characteristic (AUROC) of at least about 0.95.

In another aspect, the present disclosure provides a system for generating a reproductive hormone profile of a subject, comprising: a database comprising reference values of a reproductive hormone; a communications interface; and a computer processer operatively coupled to the database and the communications interface, wherein the computer processor is programmed to (i) to process a hair sample of the subject to generate data indicative of a presence of a reproductive hormone in the hair sample; (ii) generate an output, which output comprises the reproductive hormone profile of the project based on at least the data of (i) to the reference values of the reproductive hormone in the database; and (iii) display the output on the communications interface.

In some embodiments, the subject is a human. In some embodiments, the reproductive hormone is selected from the group consisting of dehydroepiandrosterone, estradiol, progesterone, testosterone, anti-mullerian hormone, prolactin, and vitamin D.

In some embodiments, the computer processor is further programmed to measure a presence of cortisol. In some embodiments, the computer processor is further programmed to measure a micronutrient. In some embodiments, the micronutrient is selected from the group consisting of folic acid, vitamin B12, lithium, vitamin B1, vitamin B2, vitamin B3, vitamin B5, vitamin B6, iron, iodine, phosphorus, potassium, selenium, and retinyl ester.

In some embodiments, the reproductive hormone profile is an assessment of ovarian reserve in the subject. In some embodiments, the reproductive hormone profile is an assessment of reproductive lifespan of the subject. In some embodiments, the reproductive hormone profile is an assessment of ovarian dysfunction in the subject. In some embodiments, the ovarian dysfunction is selected from the group consisting of polycystic ovary syndrome, endometriosis, anovulation, persistent follicles, and granulosa cell cancer.

In some embodiments, the reproductive hormone profile is an assessment of the subject after the subject undergoes gonadotoxic cancer treatment. In some embodiments, the reproductive hormone profile is an assessment of after the subject undergoes a complete oophorectomy. In some embodiments, the reproductive hormone profile is an assessment of the subject after the subject undergoes a partial oophorectomy. In some embodiments, the reproductive hormone profile is an assessment of metabolic syndromes, such as obesity, insulin resistance, and type 2 diabetes. In some embodiments, the reproductive hormone profile is an assessment of vitamin D deficiency in the subject. In some embodiments, the reproductive hormone profile is an assessment of thyroid dysfunction in the subject.

In some embodiments, a first hair sample is collected from the subject prior to the subject undergoing initiation of an assisted reproductive technique. In some embodiments, a second hair sample is collected from the subject subsequent to the subject undergoing initiation of an assisted reproductive technique. In some embodiments, the first hair sample or the second hair sample is selected from hair samples collected from the subject's axilla, pubic area, and head.

In some embodiments, the computer processor is further programmed to direct the processing of the hair sample to generate a solution suspected of containing the reproductive hormone and assay the solution for the presence of the reproductive hormone. In some embodiments, the computer processor is further programmed to direct the processing of the hair sample to generate a solution suspected of containing the micronutrient and assay the solution for the presence of the micronutrient.

In some embodiments, the hair sample is obtained at a location that is remotely located with respect to a location of the subject. In some embodiments, the hair sample is obtained from the remote location using a delivery service.

In some embodiments, the reference values of a reproductive hormone are obtained through a trained machine learning algorithm.

In another aspect, the present disclosure provides a system for generating a reproductive hormone profile of a subject, comprising: a database comprising reference values of an anti-mullerian hormone (AMH); a communications interface; and a computer processer operatively coupled to the database and the communications interface, wherein the computer processor is programmed to (i) receive a request to process a hair sample of the subject to generate data indicative of a presence of the AMH in the hair sample; (ii) generate an output, which output comprises the reproductive hormone profile of the subject based on at least the data of (i) to the reference values of the AMH in the database; and (iii) display the output on the communications interface.

In some embodiments, the computer processor is further programmed to measure the presence of another hormone selected from the group consisting of dehydroepiandrosterone, estradiol, progesterone, testosterone, and cortisol.

In some embodiments, the reproductive hormone profile is an assessment of ovarian reserve in the subject. In some embodiments, the reproductive hormone profile is an assessment of reproductive lifespan of the subject.

In some embodiments, the reproductive hormone profile is an assessment of ovarian dysfunction in the subject.

In some embodiments, the ovarian dysfunction is selected from the group consisting of polycystic ovary syndrome, endometriosis, anovulation, persistent follicles, and granulosa cell cancer. In some embodiments, the reproductive hormone profile is an assessment of the subject after the subject undergoes gonadotoxic cancer treatment. In some embodiments, the reproductive hormone profile is an assessment of the subject after the subject undergoes a complete oophorectomy. In some embodiments, the reproductive hormone profile is an assessment of the subject after the subject undergoes a partial oophorectomy.

In some embodiments, a first hair sample is collected from the subject prior to the subject undergoing initiation of an assisted reproductive technique. In some embodiments, a second hair sample is collected from the subject subsequent to the subject undergoing initiation of an assisted reproductive technique. In some embodiments, the first hair sample or the second hair sample is selected from hair samples collected from the subject's axilla, pubic area, and head.

In some embodiments, the computer processor is further programed to process the hair sample to generate a solution suspected of containing the AMH and assay the solution for the presence of the AMH.

In another aspect, the present disclosure provides a method of identifying or quantifying anti-mullerian hormone (AMH) in a hair sample of a subject, the method comprising: a) obtaining the hair sample of the subject; b) processing the hair sample to produce a processed sample; and c) identifying or quantifying the anti-mullerian hormone in the processed sample from b).

In another aspect, the present disclosure provides a method for identifying a reproductive disorder in a subject, comprising (a) obtaining a hair sample from the subject, (b) processing the hair sample of the subject to identify a deficiency or abundance of a reproductive hormone in the subject, and (c) electronically outputting a report indicative of the deficiency or abundance.

In another aspect, the present disclosure provides a non-transitory computer readable medium comprising machine executable code that, upon execution by one or more computer processors, implements any of the methods above or elsewhere herein.

In another aspect, the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by references to the same extent as if each individual publication, patent, or patent application was specifically and individually indicated to be incorporated by reference. To the extent publications or patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a schematic representation of a method for processing hormones, in accordance with some embodiments.

FIG. 2 shows a computer system that is programmed or otherwise configured to implement methods of the present disclosure, such as measuring hormones in samples, in accordance with some embodiments.

FIG. 3 describes a serial dilution test for anti-mullerian hormone (AMH), demonstrating the accuracy of an assay according to some embodiments of the present disclosure.

FIG. 4 shows that AMH levels in hair measured via an ELISA assay correlate with antral follicle count (AFC), in accordance with some embodiments.

FIG. 5 shows results of an assay validation for detection of AMH in hair, in accordance with some embodiments.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The terminology used herein is for the purpose of describing particular cases only and is not intended to be limiting. As used herein the singular forms “a,” “an,” and “the” are intended to include the plural forms as well, unless the context clearly dictates otherwise. Furthermore, to the extent that the terms “including,” “includes,” “having,” “has,” “with,” or variants thereof are used in either the detailed description and/or the claims, such terms are intended to be inclusive in a manner similar to the term “comprising”.

The term “about” or “nearly” as used herein generally refers to within (plus or minus) 15%, 10%, 9%, 8%, 7%, 6%, 5%, 4%, 3%, 2%, or 1% of a designated value.

The term “antibody,” as used herein, generally refers to at least the minimal portion of an antibody which may be capable of binding to an antigen, e.g., at least the variable domain of a heavy chain (VH) and the variable domain of a light chain (VL) in the context of a typical antibody produced by a B cell. Basic antibody structures in vertebrate systems are described by, e.g., Harlow et al., Antibodies: A Laboratory Manual, (Cold Spring Harbor Laboratory Press, 2nd ed. 1988), which is entirely incorporated herein by reference. Antibodies or antigen-binding fragments, variants, or derivatives may include, but are not limited to, polyclonal, monoclonal, human, humanized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domain, fragments produced by a Fab expression library. ScFv molecules are described, e.g., in U.S. Pat. No. 5,892,019. Immunoglobulin or antibody molecules encompassed by this disclosure may be of any type (e.g., IgG, IgE, IgM, IgD, IgA, and IgY), class (e.g., IgG1, IgG2, IgG3, IgG4, IgA1 and IgA2) or subclass of immunoglobulin molecule.

Antibodies or antigen-binding fragments, variants, or derivatives thereof disclosed herein may be described or specified in terms of the epitope(s) or portion(s) of an antigen, e.g., a target peptide that it recognizes or specifically binds to.

As used herein, the term “specifically binds” generally refers to a moiety that has a specific binding interaction with another moiety, such as, for example, an antibody or fragment, variant, or derivative thereof specifically bound to an epitope via its antigen-binding domain, and that the binding entails some complementarity between the antigen binding domain and the epitope.

As used herein, the term “epitope” generally refers to the specific piece of the antigen that an antibody binds to. A “linear epitope” may be formed by a continuous sequence of amino acids from the antigen. A “conformational epitope” may be composed of discontinuous sections of the antigen's amino acid sequence. Conformational epitopes interact with the antibody based on the 3-D surface features and shape or tertiary structure of the antigen. In some embodiments, epitopes stable to proteolysis are selected to avoid interference or loss of detection ability and/or sensitivity due to proteolysis.

An antibody or antigen-binding fragment, variant, or derivative thereof is the to competitively inhibit binding of a reference antibody or antigen-binding fragment to a given epitope if it preferentially binds to that epitope to the extent that it blocks, to some degree, binding of the reference antibody or antigen binding fragment to the epitope. Competitive inhibition may be determined by, for example, competition enzyme-linked immunosorbent assays (ELISAs). A binding molecule may competitively inhibit binding of the reference antibody or antigen-binding fragment to a given epitope by at least about 90%, at least about 80%, at least about 70%, at least about 60%, or at least about 50%.

The term “subject” as used herein, generally refers to any subject, such as a mammalian subject. The subject may be symptomatic with respect to a disease, disorder, or abnormal condition. The subject may be asymptomatic with respect to the disease, disorder, or abnormal condition. The subject may be undergoing diagnosis, prognosis, and/or therapy. The subject may be a human or nonhuman subject. As used herein, a subject in need thereof includes, but is not limited to, for example, women having difficulty conceiving, women at risk of infertility, women suffering from ovarian insufficiency, women suffering from premature ovarian failure, women undergoing menopause, women undergoing assisted reproductive intervention, women undergoing infertility treatment (e.g., in vitro fertilization (IVF)), women over age 35, women having diminishing functional ovarian reserve, women having ovarian dysfunction (including polycystic ovary syndrome), women undergoing gonadotoxic cancer treatment, women having complete or partial oophorectomy, women suffering from ovarian granulosa cell tumor, subjects undergoing ovarian function monitoring for childhood cancer survivors, subjects having intersex disorders, and male subjects suffering from Sertoli cell dysfunction or cancers. The subject may be having difficulty reproducing (e.g., the subject may be a woman having difficulty conceiving or a male having an issue with sperm quality (e.g., sperm count or mobility)). The subject may be male, female, or transgendered people. The subject may be a child or an adult. The child may be an individual of age from newborn to above (e.g., 18 or older, 30 or older, 40 or older, etc.).

The term “biological sample,” as used herein, generally refers to a biological sample obtained from a subject. A sample may be of any biological tissue or fluid with which biomarkers of the present disclosure may be assayed. A sample may be a “clinical sample,” e.g., a sample derived from a patient. Such samples include, but are not limited to, bodily fluids which may or may not contain cells, e.g., blood (e.g., whole blood, serum or plasma), urine, synovial fluid, saliva, breath exhalation, tears, bile, gastric fluid, vaginal secretions, breast milk, sweat, amniotic fluid, pleural fluid, tissue or fine needle biopsy samples, and archival samples with a known or measured diagnosis, treatment, and/or outcome history. Biological samples may also include sections of tissues such as frozen sections taken for histological purposes. The term “biological sample” also encompasses any material derived by processing a biological sample. Derived materials include, but are not limited to, proteins extracted from the sample. Processing of a biological sample may involve one or more of: filtration, distillation, extraction, concentration, inactivation of interfering components, addition of reagents, and the like. In some embodiments, the biological sample may be a serologic sample and may be (or may be derived from) whole blood, serum or plasma obtained from a subject. In some embodiments, the biological sample may be collected using commercially available sample collection devices, such as Super SAL™ (Oasis Diagnostics, Inc.). In some embodiments, the biological sample may be a hair sample.

As used herein, the term “control sample” refers to one, or more than one, biological samples that have been obtained from a healthy subject having normal DHEA, its sulfated form (DHEA-S), estradiol, progesterone, testosterone, and/or AMH levels for her age as measured using commercial AIA tests.

As used herein, the term “clinical laboratory” refers to a facility for the examination or processing of materials derived from a living subject, e.g., a human being. Non-limiting examples of processing include biological, biochemical, serological, chemical, immunohematological, hematological, biophysical, cytological, pathological, genetic, or other examination of materials derived from the human body for the purpose of providing information, e.g., for the diagnosis, prevention, or treatment of any disease or impairment of, or the assessment of the health of living subjects, e.g., human beings. These examinations may also include procedures to collect or otherwise obtain a sample, prepare, determine, measure, or otherwise describe the presence or absence of various substances in the body of a living subject, e.g., a human being, or a sample obtained from the body of a living subject, e.g., a human being.

As used herein, the term “point-of-care testing (POCT)” or “bedside testing” generally refers to medical diagnostic testing at or near the point of care—that is, at the time and place of patient care. Point-of-care testing allows patient diagnoses in the physician's office, an ambulance, the home, the field, or in the hospital. The results of care are timely and allow rapid treatment to the patient.

As used herein, the term “labeled” generally refers to an entity (e.g., AMH peptide, an AMH fragment or AMH peptide, or an anti-AMH antibody) that may be visualized or detected (e.g., optically detected), for example, following binding to another entity (e.g., an anti-AMH antibody). A detectable agent or moiety may be selected such that it generates a signal which may be measured and whose intensity may be related to the amount of bound entity. In array-based methods, a detectable agent or moiety may be selected such that it generates a localized signal, thereby allowing spatial resolution of the signal from each spot on the array.

Labeled polypeptides may be prepared by incorporation of or conjugation to a label, that may be directly or indirectly detectable by spectroscopic, photochemical, biochemical, immunochemical, electrical, optical, or chemical approaches, or any other suitable approach. Suitable detectable agents include, but are not limited to, various ligands, radionuclides, fluorescent dyes, chemiluminescent agents, microparticles, enzymes, colorimetric labels, magnetic labels, and haptens. In some embodiments of the present disclosure, the label used may be ruthenium to yield luminescent Ru (II) metal complexes.

As used herein, the term “level,” and grammatical variants thereof generally refers to a quantity expressed in a unit that may be obtained using any analytical method for detecting presence or expression of DHEA, DHEA-S, estradiol, progesterone, testosterone, and/or AMH in a biological sample and that indicates the presence, absence, absolute amount or concentration, relative amount or concentration, titer, expression level, ratio of measured levels, or the like, of, for, or corresponding to DHEA, DHEA-S, estradiol, progesterone, testosterone, and/or AMH in the biological sample. The exact nature of the “value” or “level” depends on the specific designs and components of the particular analytical method employed to detect DHEA, DHEA-S, estradiol, progesterone, testosterone, and/or AMH level.

As used herein, with respect to the detection of DHEA, DHEA-S, estradiol, progesterone, testosterone, and/or AMH in a sample obtained from a subject, the term “absent” or “present” generally refers to whether the level of total DHEA, DHEA-S, estradiol, progesterone, testosterone, and/or AMH may be below or above the lowest limit of quantification (LLOQ) for the analytical method used to detect DHEA, estradiol, progesterone, testosterone, and/or AMH in the biological sample. As used herein, “ultrasensitive,” detection refers to quantitative detection of total DHEA, DHEA-S, estradiol, progesterone, testosterone, and/or AMH, down to picogram (pg) levels, in a biological sample.

As used herein, the term “attached to a solid support” generally refers to the immobilization that takes place by attachment to a substrate (e.g., the surface of a well in a plate) by adsorption, covalent binding or using a specific binding pair, e.g., using the specific interaction of a suitable specific binding pair such as biotin/avidin. The solid support may comprise a protein binding surface, such as a microtiter plate, a colloidal metal particle, an iron oxide particle, a latex particle, a polymeric bead, a nanoparticle (e.g., gold nanoparticle), or Europium beads.

Hormones

AMH is a glycoprotein hormone member belonging to the transforming growth factor β (TGF-β) superfamily of proteins. These hormones may be involved in regulating cell growth and differentiation. AMH, also referred to as Mullerian Inhibiting Substance (MIS) or Mullerian-Inhibiting Hormone (MIH), is synthesized as a large precursor having an 18 amino acid signal sequence. During fetal development, AMH plays a role in sexual differentiation. It may be produced by the testicular Sertoli cells in males and by the ovarian granulosa cells in females. The Sertoli cells secrete AMH during fetal development in males, which may be essential for regression of the Mullerian ducts (primordium for the uterus, Fallopian tubes, and upper part of the vagina) and normal male reproductive tract development. Secretion of AMH by the Sertoli cells continues throughout life, with levels dropping following puberty to a relatively low value). AMH may be produced by the granulosa cells of ovarian follicles during the early stages of follicle development. Ovarian AMH production begins around birth, increases until early adulthood, and then AMH concentrations slowly decrease with increasing age until becoming undetectable about five years before menopause. The free and active form of AMH may get into the hair through the capillary network at the papilla of the hair follicle and may be absorbed by the keratinous matrix of the hair. As with the initial size and pace of follicle pool depletion, AMH levels may vary significantly in women of the same age. However, individual AMH serum concentration may accurately reflect the size of the antral follicles pool, which represents the quantity of remaining primordial follicles.

The gene encoding human AMH may be located on the short arm of chromosome 19, and the region encoding AMH may comprise 2750 nucleotides (See Cate, et al., Cell 45:685-98 (1986); Cohen-Haguenauer, et al., Cytogenet. Cell Genet. 44:2-6 (1987)), each of which is entirely incorporated herein by reference. The AMH gene may encode a 560 amino acid protein (See UniProtKB/Swiss-Prot Accession No. P03971).

AMH may bind to the extracellular domain of the AMH type II transmembrane receptor, which causes phosphorylation and activation of the type I receptor kinase and downstream signaling via Smad proteins. The Smad proteins (1, 5, or 8) may migrate into the nucleus and, in concert with other transcription factors, regulate responsive genes. Mutations in genes encoding AMH or its receptor, may affect ligand binding, signal transduction, or intracellular transport, and may often exhibit autosomal recessive segregation, causing, e.g., persistent Mullerian duct syndrome in men (Broer, et al., 2014).

In females, AMH may be a marker of ovarian reserve. This may be because the plasma levels of AMH may correlate with the number of the mature or antral follicles, which are a marker of the germinal reserve of the ovaries. Unlike other hormones, AMH is paracrine in action and may be not involved in the feedback mechanisms of the hypothalamo-pituitary-ovarian axis. In addition, AMH levels are nearly independent of the phase of the menstrual cycle because they are not affected by dominant follicle growth during the late follicular phase of the normal cycle. Thus, determination of plasma AMH may enable greater specificity and sensitivity of ovarian reserve detection over methods that determine Follicle Stimulating Hormone (FSH) together with other steroid hormones and inhibin. Other markers of ovarian aging, such as inhibin B, estradiol (E2), and FSH, are menstrual cycle dependent and constitute relatively late markers of the ongoing process of primordial follicle pool depletion. Thus, while the initial size of the follicle pool and pace of depletion may vary considerably in females (in part, reflected by the wide range of age at menopause between 40-60 years), measuring AMH concentration may provide a more accurate measure of a female's reproductive lifespan. Hair as a matrix to detect AMH may offer an integrative value of the hormone (e.g., over a previous duration of time) when compared to plasma or serum values, which shows the circulating levels of AMH at that precise moment. Hair may accumulate AMH while growing because of the capillary loop at the papilla of the hair follicle.

There are several clinical applications for measuring AMH in biological samples or biological fluids. AMH may be used as a tumor marker for tumors originating in granulosa cells because it may be produced by those cells in the ovary. With inhibin, AMH may be used as a marker of early diagnosis and response to treatment in granulosa cell tumors. The measurement of AMH may help diagnose clinical conditions that include, but are not limited to: ovarian reserve in an IVF setting, ovarian function, oocyte quality, premature ovarian failure, ovarian insufficiency, ovarian granulosa cell tumor, ovarian function for childhood cancer survivors, polycystic ovary syndrome, menopause and intersex disorders.

Dehydroepiandrosterone, also referred to as androstenolone, is an endogenous steroid hormone. It may be one of the most abundant circulating steroids in humans, and may be produced in the adrenal glands, the gonads, and the brain. Dehydroepiandrosterone (DHEA) may provide a large precursor reservoir for the intracellular production of androgens and oestrogens in non-reproductive tissues. Androgens may be responsible for the biological characteristics of males, including a deeper voice, body hair, and increased muscle mass. For example, Dihydrotestosterone (DHT) is an androgen hormone and may play an important role in the development of male sexual organs, such as penis and prostate gland. DHEA is synthesized from cholesterol via the cholesterol side-chain cleavage enzyme (CYP11A1; P450scc) and 17α-hydroxylase/17,20-lyase (CYP17A1), with pregnenolone and 17α-hydroxypregnenolone as intermediates. Dehydroepiandrosterone sulfate, abbreviated as DHEA sulfate or DHEA-S, also referred to as androstenolone sulfate, is an endogenous androstane steroid that is produced by the adrenal cortex. It is the 3β-sulfate ester and a metabolite of dehydroepiandrosterone (DHEA) that circulates in far greater relative concentrations.

Peak levels of DHEA-S may be observed around age 20, which may be followed by an age-dependent decline throughout life eventually back to prepubertal concentrations. Blood plasma or blood serum may not be able to represent long-term retrospective DHEA levels, such as it occurs for hair samples. The development of hair as a matrix for steroid detection may allow for the long-term monitoring of retrospective levels as hair accumulates circulating steroids throughout all its growth period, providing an integrative value of them. The measurement of hair steroid concentrations may make it possible to assess long-term adrenal or gonadal activity without the need of serial and continuous sampling. This may open possibilities in the study of hair samples as a matrix accumulating hydrophilic hormones.

Estradiol (E2), also spelled oestradiol, is an estrogen steroid hormone and the major female sex hormone. It may be involved in the regulation of the estrous and menstrual female reproductive cycles. Estradiol may be produced especially within the follicles of the ovaries, but also in other tissues including the testicles, the adrenal glands, fat, liver, the breasts, and the brain. Estradiol may be produced in the body from cholesterol through a series of reactions and intermediates. Estradiol, like other steroid hormones, is derived from cholesterol. After side chain cleavage and using the 45 or the 44-pathway, androstenedione is the key intermediary. A portion of the androstenedione is converted to testosterone, which in turn undergoes conversion to estradiol by aromatase. In an alternative pathway, androstenedione is aromatized to estrone, which is subsequently converted to estradiol via 17β-hydroxysteroid dehydrogenase (17β-HSD). During the reproductive years, most estradiol in women may be produced by the granulosa cells of the ovaries by the aromatization of androstenedione (produced in the theca folliculi cells) to estrone, followed by conversion of estrone to estradiol by 17β-HSD. The major pathway may involve the formation of androstenedione, which is then converted by aromatase into estrone and is subsequently converted into estradiol. In some cases, levels of DHEA, estradiol, testosterone, DHT, among other suitable hormones can provide a comprehensive overview of hormonal balance in males.

The hormones of the hypothalamic pituitary gonadal (HPG) axis may be important for coordinating ovulation, uterine endometrium development and reproductive behavior to maximize the possibility that copulation results in pregnancy. The HPG axis may regulate the release of ovarian hormones through both negative and positive feedback mechanisms. Hair may accumulate estradiol while growing because of the capillary loop at the papilla of the hair follicle.

Progesterone (P4) is an endogenous steroid and progestogen sex hormone involved in the menstrual cycle, pregnancy, and embryogenesis of humans and other species. In mammals, progesterone, like all other steroid hormones, may be synthesized from pregnenolone, which itself is derived from cholesterol. Cholesterol may undergo double oxidation to produce 22R-hydroxycholesterol and then 20α,22R-dihydroxycholesterol. This vicinal diol may be then further oxidized with loss of the side chain starting at position C22 to produce pregnenolone. This reaction may be catalyzed by cytochrome P450scc. The conversion of pregnenolone to progesterone may take place in two steps. First, the 30-hydroxyl group may be oxidized to a keto group and second, the double bond may be moved to C4, from C5 through a keto/enol tautomerization reaction.

Testosterone is the primary male sex hormone and an anabolic steroid. The largest amounts of testosterone (95% or more) may be produced by the testes in men, while the adrenal glands account for most of the remainder. Testosterone is also synthesized in far smaller total quantities in women by the adrenal glands, thecal cells of the ovaries, and, during pregnancy, by the placenta. In the testes, testosterone is produced by the Leydig cells. The male generative glands also contain Sertoli cells, which require testosterone for spermatogenesis. Like most hormones, testosterone may be supplied to target tissues in the blood where much of it may be transported bound to a specific plasma protein, sex hormone-binding globulin (SHBG).

Like other steroid hormones, testosterone may be derived from cholesterol. The biosynthesis may involve the oxidative cleavage of the side-chain of cholesterol by cholesterol side-chain cleavage enzyme (P450scc, CYP11A1), a mitochondrial cytochrome P450 oxidase with the loss of six carbon atoms to give pregnenolone. Next, two additional carbon atoms may be removed by the CYP17A1 (17α-hydroxylase/17,20-lyase) enzyme in the endoplasmic reticulum to yield a variety of C19 steroids. In addition, the 3β-hydroxyl group may be oxidized by 3β-hydroxysteroid dehydrogenase to produce androstenedione. Subsequently, the C17 keto group androstenedione may be reduced by 17β-hydroxysteroid dehydrogenase to yield testosterone. Hair as a matrix to detect testosterone may offer an integrative value of the hormone when compared to plasma or serum values. Hair may accumulate testosterone while growing because of the capillary loop at the papilla of the hair follicle. In some cases, testosterone levels in males can be measured along with FSH, prolactin, and luteinizing hormone (LH) to determine sperm health in male fertility testing.

T3 (Triiodothyronine) may be the more metabolically active hormone produced from T4. T4 is synthesized in the thyroid gland follicular cells as follows. The sodium-iodide symporter transports two sodium ions across the basement membrane of the follicular cells along with an iodine ion. This is a secondary active transporter that utilizes the concentration gradient of Na⁺ to move I⁻ against its concentration gradient. I⁻ is moved across the apical membrane into the colloid of the follicle.

Thyroperoxidase oxidizes two I⁻ to form I₂. Iodide may be non-reactive, and only the more reactive iodine may be required for the next step. The thyroperoxidase may iodinate the tyrosyl residues of the thyroglobulin within the colloid. The thyroglobulin may be synthesized in the ER of the follicular cell and secreted into the colloid. Thyroid-stimulating hormone (TSH) released from the anterior pituitary gland may bind the TSH receptor (a Gs protein-coupled receptor) on the basolateral membrane of the cell and stimulate the endocytosis of the colloid. The endocytosed vesicles may fuse with the lysosomes of the follicular cell. The lysosomal enzymes may cleave the T4 from the iodinated thyroglobulin. Hair as a matrix to detect T3 may offer an integrative value of the hormone when compared to plasma or serum values. Hair may accumulate T3 while growing because of the capillary loop at the papilla of the hair follicle.

### Reproductive Health and Sexual Wellness Markers

Thus, the present disclosure provides assays to detect to reproductive health and sexual wellness in women, men, children, and transgendered people subjects. Using these assays, a variety of markers may be identified. These include DHEA, DHEA-S, estradiol, progesterone, testosterone, AMH, Vitamin D, and micronutrients.

Assays for measuring DHEA, DHEA-S, estradiol, progesterone, testosterone, AMH, and ovarian reserve may detect total DHEA, DHEA-S, estradiol, progesterone, testosterone, and/or AMH by using antibodies. By detecting a signal with antibodies, these assays may correlate the signal to total DHEA, DHEA-S, estradiol, progesterone, testosterone, and/or AMH, and thus purport to measure total ovarian reserve.

Blood plasma or blood serum may not be able to represent long-term retrospective DHEA, DHEA-S, estradiol, progesterone, testosterone, and/or AMH levels, such as it occurs for hair samples. The development of hair as a matrix for steroid detection may allow for the long-term monitoring of retrospective levels as hair accumulates circulating steroids throughout all its growth period, providing an integrative value of them. The measurement of such markers may make it possible to assess long-term adrenal or gonadal activity without the need of serial and continuous sampling. This may open possibilities in the study of hair samples as a matrix accumulating hydrophilic hormones, such as DHEA, DHEA-S, estradiol, progesterone, testosterone, and/or AMH, while the hair is growing. Hair may accumulate DHEA, DHEA-S, estradiol, progesterone, testosterone, and/or AMH during all the hair growth period, and the levels detected in hair may indicate the amount of protein circulating in blood, and therefore getting all the tissues of the body, including the skin and its keratinous derivatives (such as the capillary network of the hair). This may possibly provide a global measure of DHEA, DHEA-S, estradiol, progesterone, testosterone, and/or AMH levels during the period hair is growing (weeks to months due to an estimated growth of 1 mm/month). DHEA, DHEA-S, estradiol, progesterone, testosterone, and/or AMH levels in hair may have the potential of being an integrative measure of the follicle ovarian reserve, which in theory may be better than plasma values, since hair DHEA, DHEA-S, estradiol, progesterone, testosterone, and/or AMH measurements lack influences of circadian rhythms or specific acute physiological situations affecting blood composition. The interpretation of such values may be correlated with plasma levels. For example, DHEA-S level measured from a hair sample can be compared to such level obtained from plasma from the same testing subject. The fold changes between the hair and plasma samples may be helpful to provide additional information to medical workers. Further, the interpretation of such values may be correlated with antral follicular count.

In an aspect, the present disclosure provides a procedure which demonstrates that DHEA, DHEA-S, estradiol, progesterone, testosterone, and/or AMH may be linked to the keratinous matrix of the hair and its levels may be different among the individuals analyzed.

There remains a need to correlate hormone levels with the remaining functional follicular cells. This need represents a significant problem across a variety of clinical situations affected by diminishing functional ovarian reserve, including, e.g., infertility treatment (including in vitro fertilization (IVF)), the forecasting of reproductive lifespan, ovarian dysfunction (including polycystic ovary syndrome), gonadotoxic cancer treatment, and complete or partial oophorectomy. By identifying the continuous DHEA, DHEA-S, estradiol, progesterone, testosterone, and/or AMH follicular production linked to hair samples and how these levels may be related to the functional follicular ovarian reserve, detecting and quantifying hormone levels may help improve success rates of, among other things, conception and assisted reproductive techniques.

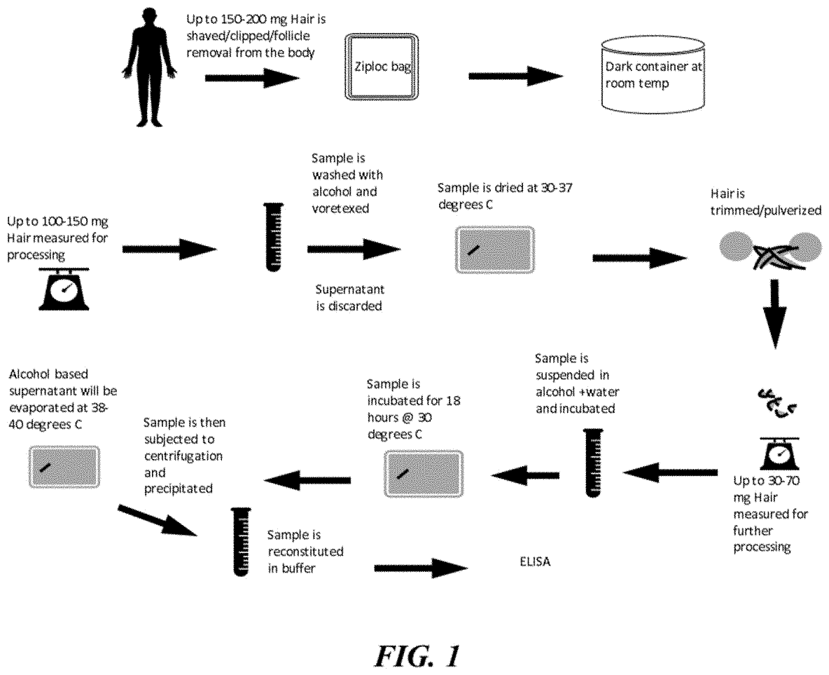

The present disclosure provides compositions, methods, systems, devices, platforms, and kits for detecting and/or quantifying the presence of DHEA, DHEA-S, estradiol, progesterone, testosterone, and/or AMH in a biological sample. In some embodiments, the present disclosure provides compositions and methods for detecting and/or quantifying the presence of DHEA, DHEA-S, estradiol, progesterone, testosterone, and/or AMH (as shown in FIG. 1). Further, the disclosed compositions and methods may be used to measure various hormone levels in a test subject before, after, and during a hormone therapy. In some embodiments, the present disclosure provides antibodies for detecting and/or quantifying DHEA, DHEA-S, estradiol, progesterone, testosterone, and/or AMH. Similarly, the disclosed antibodies may be used to measure various hormone levels in a test subject before, after, and during a hormone therapy.

The detection and/or quantification of DHEA, DHEA-S, estradiol, progesterone, testosterone, and/or AMH in hair, as disclosed herein, may provide advantages over current detection in plasma or serum. DHEA, DHEA-S, estradiol, progesterone, testosterone, and/or AMH may be detected by enzyme immunoassay (EIA) and other detection tests. The compositions and methods of the present disclosure may recognize hair DHEA, DHEA-S, estradiol, progesterone, testosterone, and/or AMH, through a protocol of extraction. Hormone levels may provide a key tool and/or biomarker to better understand DHEA, DHEA-S, estradiol, progesterone, testosterone, and/or AMH biology, and to elucidate the role of DHEA, DHEA-S, estradiol, progesterone, testosterone, and/or AMH in physiology and pathology.

As described herein, the extraction procedure of DHEA, DHEA-S, estradiol, progesterone, testosterone, and/or AMH from hair may be successful in detecting amounts as low as nanogram (ng) amounts of DHEA, DHEA-S, estradiol, progesterone, testosterone, and/or AMH per ml of hair extract. Thus, the procedure described herein may provide an improved approach to evaluate egg quality or functional ovarian reserve and may be used to tailor treatment options (for example, planning regimen for assisted reproduction) on that basis.

In some embodiments, the present disclosure provides an enzyme immunoassay (EIA) (for example, competitive EIA), for detecting and/or quantifying hormone levels. In some embodiments, the present disclosure provides a rapid, sensitive or ultrasensitive EIA for detecting and/or quantifying hormone levels. For example, the disclosed method and other suitable method can be used to monitor various hormone levels in transgendered people receiving hormone therapies, such as masculinizing hormone therapies (reducing estradiol and increasing testosterone) and feminizing hormone therapies (increasing estradiol level and reducing testosterone). Further, the disclosed method and other suitable method can be used to measure LH, FSH, oestradiol, testosterone, and other hormones to diagnose puberty delay in children.

In some embodiments, the present disclosure provides a portable or handheld device, e.g., a smartphone or a smartphone-controlled handheld device, which may be configured or adapted for use with any embodiment of the present disclosure. In some embodiments, the present disclosure provides a packaged article, e.g., an article of manufacture, such as a system, an assay and/or detection or diagnostic kit, comprising any of the components (e.g., composition comprising one or more antibodies and/or DHEA, DHEA-S, estradiol, progesterone, testosterone, and/or AMH peptides/peptide fragments, controls, calibrators) of the disclosure, optionally with a label(s) and/or with instructions for use. Such label(s) may include components and/or compatible analytes. Such instructions may include directing or promoting, including advertising, use of the article of manufacture.

In an aspect, the present disclosure relates to a method of manufacturing an article of manufacture comprising any of the components described herein, packaging the composition to obtain an article of manufacture and instructing, directing or promoting the use of the article of manufacture for any of the uses described herein. Such instructing, directing or promoting may include advertising.

### Methods of Detecting Reproductive Health and Sexual Wellness Markers

An aspect of the present disclosure provides a method for extraction of DHEA, DHEA-S, estradiol, progesterone, testosterone, and/or AMH from hair samples for detecting and/or quantifying the presence of DHEA, DHEA-S, estradiol, progesterone, testosterone, and/or AMH in a biological sample. In some embodiments, the biological sample is a hair sample. In some embodiments, the biological sample comprise a whole blood sample, serum sample, a plasma sample, a saliva sample, a follicular fluid sample, a tissue sample, a hair sample, or combinations thereof.

An aspect of the present disclosure provides a method comprising: (a) obtaining a biological sample from a subject (for example hair or plasma or serum sample); (b) extracting DHEA, DHEA-S, estradiol, progesterone, testosterone, and/or AMH from the matrix; (c) detecting and/or quantifying total DHEA, estradiol, progesterone, testosterone, and/or AMH in the sample; and (d) correlating values in the different biological samples. In some embodiments, (c) comprises quantifying total DHEA, estradiol, progesterone, testosterone, and/or AMH in the sample. In some cases, multiple samples may be collected from an individual. For example, a combination of any two or more of whole blood sample, serum sample, plasma sample, saliva sample, follicular fluid sample, tissue sample, and hair sample may be collected from the same individual. Expression levels of DHEA, DHEA-S, estradiol, progesterone, testosterone, and/or AMH may be measured and quantified from each different kind of sample from the same individual (see example 4). Multiple consecutive samples from the same individual may be used to create algorithms that may better predict ovarian reserves, pre-menopause, etc.

In some embodiments, the DHEA, DHEA-S, estradiol, progesterone, testosterone, and/or AMH quantification may be performed using a polyclonal antibody, a monoclonal antibody, or a combination thereof. In some embodiments, the antibody may be immobilized on or bound to a solid support. In some embodiments, the solid support comprises a protein binding surface selected from a microtiter plate, a colloidal metal particle, an iron oxide particle, a latex particle, a polymeric bead, and a nanoparticle (e.g., gold nanoparticles). In some embodiments, first and second antibodies are used in a single portion of the biological sample or in separate portions of the biological sample. In some embodiments, detecting and/or quantifying of the (c) of disclosed methods may be performed in a single portion of the biological sample or in separate portions of the biological sample. In some embodiments, the total DHEA, DHEA-S, estradiol, progesterone, testosterone, and/or AMH levels are detected using a chemiluminescent agent, a colorimetric agent, an energy transfer agent, an enzyme, a fluorescent agent, a radioisotope, or a combination thereof.

An aspect of the present disclosure provides a method for determining or assessing ovarian reserve in a subject comprising: (a) extracting DHEA, DHEA-S, estradiol, progesterone, testosterone, and/or AMH from a biological sample (for example hair or plasma or serum) obtained from the subject; (b) detecting and/or quantifying DHEA, DHEA-S, estradiol, progesterone, testosterone, and/or AMH in the biological sample; and (c) correlating levels in hair or plasma or serum, to indicate if the subject's ovarian reserve may be altered and/or different compared to the levels of a control subject. In some embodiments, the method provides for determining or assessing the quality of the ovarian reserve in a subject comprising performing one or more of the above operations (a) through (c). In some embodiments, (b) comprises quantifying DHEA, DHEA-S, estradiol, progesterone, testosterone, and/or AMH.

In some embodiments, the biological sample may be collected before initiation of the assisted reproductive technique. In some embodiments, the biological sample may be collected after initiation of the assisted reproductive technique. In some embodiments, the biological sample may be collected before initiation of the assisted reproductive technique and after initiation of the assisted reproductive technique.

An aspect of the present disclosure provides, in any of the disclosed methods, the detecting and/or quantifying of DHEA, DHEA-S, estradiol, progesterone, testosterone, and/or AMH in the biological sample using an antibody.

In an aspect, the present disclosure provides a method of measuring DHEA, DHEA-S, estradiol, progesterone, testosterone, and/or AMH in hair from a subject, the method comprising: a) obtaining hair from a subject; b) processing the hair to produce a sample; and c) quantifying the DHEA, DHEA-S, estradiol, progesterone, testosterone, and/or AMH in the sample from b).

A method for generating a reproductive hormone profile of a subject, may comprise: (a) obtaining a hair sample from the subject; (b) processing the hair sample from the human subject to generate data indicative of a presence of a reproductive hormone in the hair sample; and (c) inputting at least the data of (b) to a reproductive hormone classifier to generate the reproductive hormone profile of the human subject.

The reproductive hormone may be selected from the group consisting of dehydroepiandrosterone, estradiol, progesterone, testosterone, anti-mullerian hormone, prolactin, and vitamin D. The method may further comprise measuring a presence of cortisol.

The method may further comprise measuring a micronutrient. The micronutrients may be selected from the group consisting of water-soluble vitamins, fat soluble vitamins, macrominerals or elements, trace minerals or elements, trace non-metal elements, ultra-trace minerals or elements, other essential compounds, conditionally essential nutrients, carotenoids, fats or lipids, and proteins. The micronutrient may be selected from the group consisting of thiamine, riboflavin, niacin, pantothenic acid, pyridoxine (pyridoxal-5-phosphate or pyridoxamine), biotin, folate or folic acid, cobalamin, ascorbic acid, retinol, calciferol (ergocalciferol or cholacalciferol), tocopherol (or tocotrienol), naphthoquinoids, phylloquinone (K1), manaquinione (K2), calcium, chloride, magnesium, phosphorus, potassium, sodium, sulfur, copper, fluoride, iron, manganese, nickel, zinc, iodine, selenium, boron, bromine, chromium, cobalt, molybdenum, choline, inositol, taurine, arginine, glutamine, alpha carotene, beta carotene, cryptoxanthin, lutein, lycopene, zeaxanthin, omega-3 fatty acids such as EPA, alpha-linolenic acid, eicosapentaenoic acid, docosahexaenoic acid or DHA, omega-6 fatty acids such as linoleic acid, phenylalanine, valine, threonine, tryptophan, methionine, leucine, isoleucine, lysine, and histidine. The micronutrient may be selected from the group consisting of folic acid, vitamin B12, lithium, vitamin B1, vitamin B2, vitamin B3, vitamin B5, vitamin B6, iron, iodine, phosphorus, potassium, selenium, and retinyl ester.

The reproductive hormone profile may be an assessment of ovarian reserve in the subject. The reproductive hormone profile may be an assessment of reproductive lifespan of the subject. The reproductive hormone profile may be an assessment of ovarian dysfunction of the subject. The ovarian dysfunction may be selected from the group consisting of polycystic ovary syndrome, endometriosis, anovulation, persistent follicles, and granulosa cell cancer.

The reproductive hormone profile may be an assessment of the subject after the subject undergoes gonadotoxic cancer treatment. The reproductive hormone profile may be an assessment of the subject after the subject undergoes a complete oophorectomy. The reproductive hormone profile may be an assessment of the subject after the subject undergoes a partial oophorectomy. The reproductive hormone profile may be an assessment of metabolic syndromes, such as obesity, insulin resistance, and type 2 diabetes. The reproductive hormone profile may be an assessment of vitamin D deficiency in the subject. The reproductive hormone profile may be an assessment of thyroid dysfunction in the subject.

The method may further comprise obtaining a first hair sample from the subject prior to the subject undergoing initiation of an assisted reproductive technique. The method may further comprise obtaining a second hair sample from the subject subsequent to the subject undergoing initiation of an assisted reproductive technique. The first hair sample and the second hair sample may be selected from hair samples collected from the subject's axilla, pubic area, and head.

The method may include operation (b) which further comprises using the hair sample to generate a solution suspected of containing the reproductive hormone and assaying the solution for the presence of the reproductive hormone. The method may include operation (b) which further comprises using the hair sample to generate a solution suspected of containing the micronutrient and assaying the solution for the presence of the micronutrient.

The hair sample may be obtained at a location that is remotely located with respect to a location of the subject. The hair sample may be obtained from the remote location using a delivery service.

The reproductive hormone classifier may be a trained machine learning algorithm. The method may include operation (c) which further comprises generating an electronic report having the reproductive hormone profile of the subject. The electronic report may be provided for display on an electronic device of the subject.

A method for generating a reproductive hormone profile of a subject, may comprise: (a) obtaining a hair sample from the subject; (b) processing the hair sample from the subject to generate data indicative of a presence of an anti-mullerian hormone (AMH) in the hair sample; and (c) using at least the data in (b) to generate the reproductive hormone profile of the subject.

The method may further comprise measuring a presence of another hormone selected from the group consisting of dehydroepiandrosterone, estradiol, progesterone, testosterone, and cortisol.

The reproductive hormone profile may be an assessment of ovarian reserve in the subject. The reproductive hormone profile may be an assessment of reproductive lifespan of the subject. The reproductive hormone profile may be an assessment of ovarian dysfunction of the subject. The ovarian dysfunction may be selected from the group consisting of polycystic ovary syndrome, endometriosis, anovulation, persistent follicles, and granulosa cell cancer.

The reproductive hormone profile may be an assessment of the subject after the subject undergoes gonadotoxic cancer treatment. The reproductive hormone profile may be an assessment of the subject after the subject undergoes a complete oophorectomy. The reproductive hormone profile may be an assessment of the subject after the subject undergoes a partial oophorectomy.

The method may further comprise obtaining a first hair sample from the subject prior to the subject undergoing initiation of an assisted reproductive technique. The method may further comprise obtaining a second hair sample from the subject subsequent to the subject undergoing initiation of an assisted reproductive technique. The first hair sample and the second hair sample may be selected from hair samples collected from the subject's axilla, pubic area, and head.

The method may include operation (b) which further comprises using the hair sample to generate a solution suspected of containing the AMH and assaying the solution for the presence of the AMH. The hair sample may be obtained at a location that is remotely located with respect to a location of the subject. The hair sample may be obtained from the remote location using a delivery service.

The reproductive hormone classifier may be a trained machine learning algorithm. The method may include operation (c) which further comprises generating an electronic report having the reproductive hormone profile of the subject. The electronic report may be provided for display on an electronic device of the subject.

A method of identifying or quantifying anti-mullerian hormone in a hair sample of a subject may comprise: a) obtaining the hair sample of the subject; processing the hair sample to produce a processed sample; and identifying or quantifying the anti-mullerian hormone in the processed sample from b).

A method for identifying a reproductive disorder in a subject may comprise (a) obtaining a hair sample from the subject, (b) processing the hair sample of the subject to identify a deficiency or abundance of a reproductive hormone in the subject, and (c) electronically outputting a report indicative of the deficiency or abundance.

In a specific embodiment, DHEA, estradiol, progesterone, testosterone, and/or AMH may be detected and/or quantified in the biological sample using an immunoassay. In some embodiments, the immunoassay uses recombinant or synthetically prepared DHEA, estradiol, progesterone, testosterone, and/or AMH proteins or polypeptides.

Embodiments as described herein may be used in various types of immunoassays, which may include a competitive type of immunoassay. Examples of immunoassays that may be competitive include an enzyme immunoassay or enzyme-linked immunosorbent assay (EIA or ELISA), a fluorescent immunoassay, a radiometric or radioimmunoassay (RIA), a magnetic separation assay (MSA), a lateral flow test/assay (LFT), a diffusion immunoassay, an immunoprecipitation assay, an immunosorbent or “antigen-down” assay using an analyte bound to a solid support, and an agglutination assay.

In some embodiments, the immunoassay comprises an EIA (e.g., quantitative EIA) to detect and/or quantify DHEA, DHEA-S, estradiol, progesterone, testosterone, and/or AMH in biological fluids (e.g., blood, serum, saliva), in which a first antibody (“capture antibody”) may be attached to a solid support. DHEA, DHEA-S, estradiol, progesterone, testosterone, and/or AMH from a sample or standard (“unlabeled antigen), and a labeled antigen (e.g., recombinant DHEA, DHEA-S, estradiol, progesterone, testosterone, and/or AMH peptide, or synthetic DHEA, DHEA-S, estradiol, progesterone, testosterone, and/or AMH peptide), which may be conjugated to a detection label/tag, are allowed to bind to the capture antibody, and detected either by an enzymatic reaction, an electrochemiluminescent (ECL) reaction, radioactivity, or other detection method.

In some embodiments, immunoassay comprises a competitive EIA, which may be a quantitative competitive EIA. In one such assay, a sample contains an unknown amount of analyte to be measured. The analyte may be also referred to as an antigen. The antigen may be in the sample or a component of the detection step. The sample (comprising an “unlabeled” antigen) may be spiked with a known or fixed amount of “labeled” antigen. The spiked sample may then be incubated with an antibody that binds to the antigen, so that the unlabeled antigen in the sample and the labeled antigen added to the sample “compete” for binding (e.g., bind competitively) to the available antibody binding sites. More or less of the labeled antigen may be able to bind to the antibody binding sites, depending on the relative concentration of the unlabeled antigen present in the sample. Accordingly, when the amount of labeled antigen bound to the antibody may be measured, it may be inversely proportional to the amount of unlabeled antigen in the sample. The amount of antigen (e.g., total DHEA, DHEA-S, estradiol, progesterone, testosterone, or AMH) in the original sample may then be calculated based on the amount of labeled antigen measured, e.g., using standard methods.

In certain embodiments, the immunoassay comprises a sandwich immunoassay, e.g., an enzyme-linked immunosorbent assay (ELISA) or a sandwich ECL assay. In certain embodiments, the present disclosure provides immunometric, “two-site” or “sandwich” immunoassays, wherein the analyte may be bound to or sandwiched between two antibodies that bind to different epitopes on the analyte. Representative examples of such immunoassays include enzyme immunoassays or enzyme-linked immunosorbent assays (EIA or ELISA), immunoradiometric assays (IRMA), fluorescent immunoassays, lateral flow assays, diffusion immunoassays, immunoprecipitation assays, and magnetic separation assays (MSA). Further, chemiluminiscence and electrochemiluminiscence techniques may also be employed for the hormone determination, such as High-Performance Liquid Chromatography and Mass Spectrometry (HPLC-MS/LC-MS) for assay validation purposes.

In one such assay, a first antibody, which may be described as the “capture” antibody, may be bound to a solid support, such as a protein binding surface, colloidal metal particles, iron oxide particles, latex particles or polymeric beads. The capture antibody may be bound to or coated on a solid support. Alternatively, the capture antibody may be coupled with a ligand that may be recognized by an additional antibody that may be bound to or coated on a solid support. Binding of the capture antibody to the additional antibody via the ligand then indirectly immobilizes the capture antibody on the solid support. An example of such a ligand may be fluorescein. The second antibody, which may be described as the “detection” antibody, may be coupled with a label, which may comprise a chemiluminescent agent, a calorimetric agent, an energy transfer agent, an enzyme, a fluorescent agent or a radioisotope. The detection antibody may be coupled with or conjugated with a label. The label may comprise a first protein such as biotin coupled with the second antibody, and a second protein such as streptavidin that may be coupled to an enzyme. The second protein binds to the first protein. The enzyme produces a detectable signal when provided with substrate(s), so that the amount of signal measured corresponds to the amount of second antibody that may be bound to the analyte. Horseradish peroxidase is an example of such an enzyme. Other possible substrates include, but are not limited to, TMB (3,3′, 5,5′-tetramethyl benzidine), OPD (o-phenylene diamine), and ABTS (2,2′-azino-bis (3-ethylbenzthiazoline-6-sulfonic acid). If the detection molecule is tagged with biotin, then enzyme-conjugated streptavidin is added, unbound streptavidin is washed away, and a substrate is added which provides a colorimetric reaction that may be read, e.g., on a spectrophotometer.

In certain embodiments, the present disclosure provides a competitive immunoassay where an antibody that binds to the sample (analyte or antigen) may be coupled with or conjugated with a ligand, wherein the ligand binds to an additional antibody added to the sample. One example of such a ligand includes fluorescein. The additional antibody may be bound to a solid support. The additional antibody binds to the ligand coupled with the antibody that binds in turn to the analyte or alternatively to the labeled analyte, forming a mass complex which may allow isolation and measurement of the signal generated by the label coupled with the labeled analyte. In another type of competitive immunoassay, the analyte to be measured may be bound to a solid support and incubated with both an antibody that binds to the analyte and a sample containing the analyte to be measured. The antibody binds to either the analyte bound to the solid support or to the analyte in the sample, in relative proportions depending on the concentration of the analyte in the sample. The antibody that binds to the analyte bound to the solid support may then be bound to another antibody that may be coupled with a label. The amount of signal generated from the label may then be detected to measure the amount of antibody that bound to the analyte bound to the solid support. Such a measurement may be inversely proportional to the amount of analyte present in the sample. Such an assay may be used in a microtiter plate format. Another immunoassay that may be competitive includes an agglutination assay. In such an assay, the analyte to be measured in the sample competes with analyte that may be bound to a first solid support particle, such as Ficoll.

The antibody may be bound to or coated on a second solid support particle. Cross-binding or agglutination between the particles occurs as the analyte molecules bound to the first solid support particles bind to the antibody molecules bound to the second solid support particles, to form clumps of co-agglutination lattice. Alternatively, the antibody molecules bind to the free analyte in the sample, so that the amount of agglutination may be inversely proportional to the amount of analyte in the sample. The amount of agglutination may be measured using various techniques, such as spectrophotometry.

The detection molecule (antibody or a DHEA, DHEA-S, estradiol, progesterone, testosterone, and/or AMH peptide) may be directly labeled with an enzyme, e.g., horseradish peroxidase or alkaline phosphatase, or may be labeled with a tag that may allow an enzyme to bind. Examples of enzyme labels include, but are not limited to, alkaline phosphatase, peroxidase, and galactosidase. For example, the detection antibody may be conjugated to biotin, and the enzyme attached in a subsequent operation by allowing enzyme-conjugated streptavidin to bind to the biotin tag. Alternatively, the detection molecule may be conjugated to, e.g., a chemiluminescent, fluorescent, or electrochemiluminescent (ECL) tag. An example of the latter may be a ruthenium chelate. Following incubation, the plate may then be washed to remove any unbound detection molecule.

Based on comparison to reference samples with corresponding known outcomes, a “threshold level” may be determined, and test samples that fall above that threshold level may indicate, for example, that the patient from whom the sample was obtained may benefit from a specific treatment option.

In some embodiments, the results may be expressed as a ratio with the control samples to determine a variation in the subject's total dehydroepiandrosterone (DHEA), dehydroepiandrosterone sulfate (DHEA-S), estradiol, progesterone, testosterone, and/or AMH levels compared to the control levels. According to this embodiment, the control sample may be a matched pair with the patient sample, e.g., one or more of whole blood if the patient sample is whole blood, serum if the patient sample is serum, plasma if the patient sample is plasma, or saliva if the patient sample is saliva.

In an aspect, the present disclosure provides a composition comprising a single antibody (polyclonal or monoclonal), which binds specifically and/or with high affinity to stable epitopes on DHEA, DHEA-S, estradiol, progesterone, testosterone, and/or AMH. In some embodiments, the single antibody may be bound to a solid support or be bound to or coated on to a solid support.

The present disclosure provides a composition comprising a first antibody and a second antibody, wherein the first antibody binds to a first epitope and the second antibody binds to a second epitope in a region of DHEA, DHEA-S, estradiol, progesterone, testosterone, and/or AMH. Such antibodies may be useful in immunoassays to measure an amount of DHEA, DHEA-S, estradiol, progesterone, testosterone, and/or AMH in a sample. The first and second antibodies may be monoclonal antibodies. Such antibodies may bind to epitopes in the region of DHEA, DHEA-S, estradiol, progesterone, testosterone, and/or AMH. The first epitope to which the first antibody binds may be different from the second epitope to which the second antibody binds, so that binding of one antibody to its epitope does not interfere with the binding of the other antibody. The first and second antibodies may also comprise antibody fragments.

In some embodiments, there may be a composition for measuring an amount of DHEA, DHEA-S, estradiol, progesterone, testosterone, and/or AMH in a sample comprising: a first antibody and a second antibody that bind to a region of DHEA, DHEA-S, estradiol, progesterone, testosterone, and/or AMH, wherein the first antibody binds to a first epitope and the second antibody binds to a second epitope in the region of DHEA, DHEA-S, estradiol, progesterone, testosterone, and/or AMH; a solid support coupled with the first antibody; and a label coupled with the second antibody. In some embodiments, the solid support comprises a protein binding surface, which may include, but may be not limited to, a microtiter plate, a colloidal metal particle, an iron oxide particle, a latex particle or a polymeric bead. The label may comprise a chemiluminescent agent, a calorimetric agent, an energy transfer agent, an enzyme, a fluorescent agent or a radioisotope, or another type of Such as compositions as disclosed herein may comprise kits useful for measuring total AMH in a sample.

In some embodiments, there may be a method for measuring an amount of an DHEA, DHEA-S, estradiol, progesterone, testosterone, and/or AMH form in a sample containing DHEA, DHEA-S, estradiol, progesterone, testosterone, and/or AMH comprising: binding a first antibody to an DHEA, DHEA-S, estradiol, progesterone, testosterone, and/or AMH, wherein the first antibody binds to a first epitope in SDMF; binding a second antibody to the DHEA, DHEA-S, estradiol, progesterone, testosterone, and/or AMH, wherein the second antibody binds to a second epitope in SDMF, thereby creating an amount of bound second antibody; measuring the amount of bound second antibody; and calculating the amount of SDMF in the sample. In some embodiments, a solid support may be bound to the first antibody.

In some embodiments, the compositions and methods of the present disclosure are adapted for use with commercially available test platforms, such as VerOFy® from Oasis Diagnostics, Inc. VerOFy® is a platform technology that combines rapid and standardized saliva [oral fluid] collection with high quality immunochromatographic test strips providing a system for delivery of immediate results in field or point-of-care locations. LIAM™ (Light Image Analysis Module) is a portable scanning module for use with the VerOFy® technology. LIAM™ is designed to quantify results from a VerOFy® Rapid, Oral Fluid Test. Following quantification, the LIAM™ archives a limited number of results, and also offers the ability to transfer files directly to a smart phone or Bluetooth capable device. LIAM™ is battery powered, light, hand-held, and is capable of operating in hard to reach field locations.

### Computer Systems

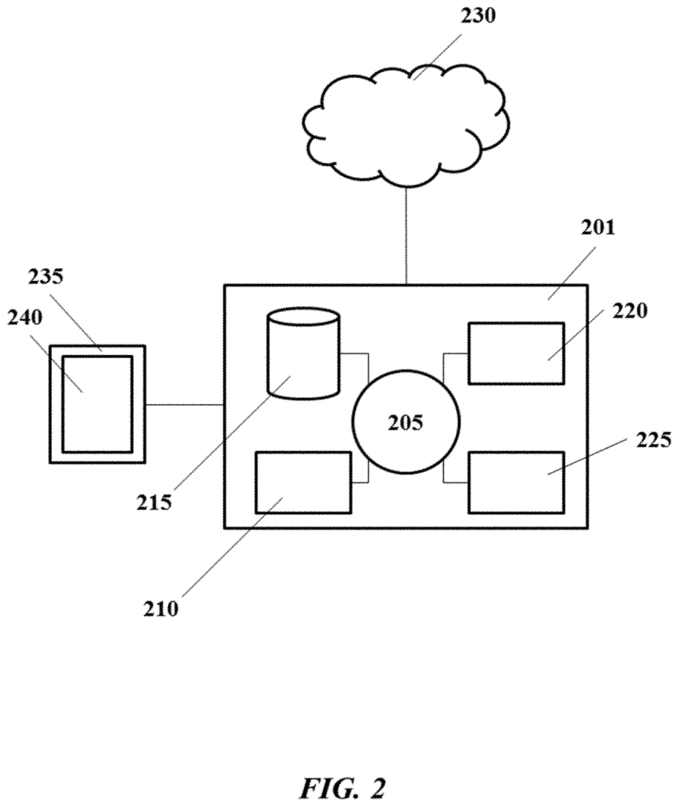

The present disclosure provides computer systems that are programmed to implement methods of the disclosure. FIG. 2 shows a computer system 201 that is programmed or otherwise configured to measure AMH in samples. The computer system 201 can regulate various aspects of the methods of the present disclosure, such as, for example, the extraction and detection of DHEA, DHEA-S, estradiol, progesterone, testosterone, and/or AMH in a sample. The computer system 201 can be an electronic device of a user or a computer system that is remotely located with respect to the electronic device. The electronic device can be a mobile electronic device.

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), such as a motherboard. The storage unit 215 can be a data storage unit (or data repository) for storing data. The computer system 201 can be operatively coupled to a computer network (“network”) 230 with the aid of the communication interface 220. The network 230 can be the Internet, an internet and/or extranet, or an intranet and/or extranet that is in communication with the Internet. The network 230 in some cases is a telecommunication and/or data network. The network 230 can include one or more computer servers, which can enable distributed computing, such as cloud computing. The network 230, in some cases with the aid of the computer system 201, can implement a peer-to-peer network, which may enable devices coupled to the computer system 201 to behave as a client or a server.

The CPU 205 can execute a sequence of machine-readable instructions, which can be embodied in a program or software. The instructions may be stored in a memory location, such as the memory 210. The instructions can be directed to the CPU 205, which can subsequently program or otherwise configure the CPU 205 to implement methods of the present disclosure. Examples of operations performed by the CPU 205 can include fetch, decode, execute, and writeback.

The CPU 205 can be part of a circuit, such as an integrated circuit. One or more other components of the system 201 can be included in the circuit. In some cases, the circuit is an application specific integrated circuit (ASIC).

The storage unit 215 can store files, such as drivers, libraries and saved programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet.

The computer system 201 can communicate with one or more remote computer systems through the network 230. For instance, the computer system 201 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine-readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display 235 that comprises a user interface (UI) 240 for providing, for example, measurements of the reproductive hormone (e.g., DHEA, DHEA-S, estradiol, progesterone, testosterone, and/or AMH). Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 205. The algorithm can, for example, determine the levels of DHEA, DHEA-S, estradiol, progesterone, testosterone, and/or AMH in a biological sample.

A system for generating a reproductive hormone profile of a subject may comprise: a database comprising reference values of a reproductive hormone; a communications interface; and a computer processer operatively coupled to the database and the communications interface, wherein the computer processor is programmed to (i) to process a hair sample from a subject to generate data indicative of a presence of a reproductive hormone in the hair sample; (ii) generate an output, which output comprises a reproductive hormone profile of the project based on at least the data of (i) to the reference values of the reproductive hormone in the database; and (iii) display the output on the communications interface.

The reproductive hormone may be selected from the group consisting of dehydroepiandrosterone, estradiol, progesterone, testosterone, anti-mullerian hormone, prolactin, and vitamin D. The computer processor may be further programed to measure a presence of cortisol.

The computer processor may be further programed to measure a micronutrient. The micronutrient may be selected from the group consisting of folic acid, vitamin B12, lithium, vitamin B1, vitamin B2, vitamin B3, vitamin B5, vitamin B6, iron, iodine, phosphorus, potassium, selenium, and retinyl ester.

The reproductive hormone profile may be an assessment of ovarian reserve in the subject. The reproductive hormone profile may be an assessment of reproductive lifespan of the subject. The reproductive hormone profile may be an assessment of ovarian dysfunction in the subject. The ovarian dysfunction may be selected from the group consisting of polycystic ovary syndrome, endometriosis, anovulation, persistent follicles, and granulosa cell cancer.

The reproductive hormone profile may be an assessment of the subject after the subject undergoes gonadotoxic cancer treatment. The reproductive hormone profile may be an assessment of after the subject undergoes a complete oophorectomy. The reproductive hormone profile may be an assessment of the subject after the subject undergoes a partial oophorectomy. The reproductive hormone profile may be an assessment of metabolic syndromes, such as obesity, insulin resistance, and type 2 diabetes. The reproductive hormone profile may be an assessment of vitamin D deficiency in the subject. The reproductive hormone profile may be an assessment of thyroid dysfunction in the subject.

In some embodiments of the system, a first hair sample is collected from the subject prior to the subject undergoing initiation of an assisted reproductive technique. In some embodiments of the system, a second hair sample is collected from the subject subsequent to the subject undergoing initiation of an assisted reproductive technique. The first hair sample or the second hair sample may be selected from hair samples collected from the subject's axilla, pubic area, and head.

The computer processor may be further programmed to direct the processing of the hair sample to generate a solution suspected of containing the reproductive hormone and assay the solution for the presence of the reproductive hormone. The computer processor may be further programmed to direct the processing of the hair sample to generate a solution suspected of containing the micronutrient and assay the solution for the presence of the micronutrient.

The hair sample may be obtained at a location that is remotely located with respect to a location of the subject. The hair sample may be obtained from the remote location using a delivery service. The reference values of a reproductive hormone may be obtained through a trained machine learning algorithm.

A system for generating a reproductive hormone profile of a subject may comprise: a database comprising reference values of a marker; a communications interface; and a computer processer operatively coupled to the database and the communications interface, wherein the computer processor is programmed to (i) receive a request to process a hair sample from a human subject to generate data indicative of a presence of the marker in the hair sample; (ii) generate an output, which output comprises a reproductive hormone profile of the project based on at least the data of (i) to the reference values of the marker in the database; and (iii) display the output on the communications interface.

The marker being analyzed by the system may be a marker of reproductive health and sexual wellness. Such a marker may include DHEA, DHEA-S, estradiol, progesterone, testosterone, AMH, Vitamin D, and/or micronutrients.

A system for generating a reproductive hormone profile of a subject may comprise: a database comprising reference values of an anti-mullerian hormone; a communications interface; and a computer processer operatively coupled to the database and the communications interface, wherein the computer processor is programmed to (i) receive a request to process a hair sample from a human subject to generate data indicative of a presence of the AMH in the hair sample; (ii) generate an output, which output comprises a reproductive hormone profile of the project based on at least the data of (i) to the reference values of the AMH in the database; and (iii) display the output on the communications interface.

The computer processor may be further programmed to measure the presence of another hormone selected from the group consisting of dehydroepiandrosterone, estradiol, progesterone, testosterone, and cortisol.

The reproductive hormone profile may be an assessment of ovarian reserve in the subject. The reproductive hormone profile may be an assessment of reproductive lifespan of the subject. The reproductive hormone profile may be an assessment of ovarian dysfunction in the subject. The ovarian dysfunction may be selected from the group consisting of polycystic ovary syndrome, endometriosis, anovulation, persistent follicles, and granulosa cell cancer.

The reproductive hormone profile may be an assessment of the subject after the subject undergoes gonadotoxic cancer treatment. The reproductive hormone profile may be an assessment of the subject after the subject undergoes a complete oophorectomy. The reproductive hormone profile may be an assessment of the subject after the subject undergoes a partial oophorectomy.

The hair sample of the human subject may be a first hair sample collected from the subject prior to the subject undergoing initiation of an assisted reproductive technique. The hair sample of the human subject may be a second hair sample collected from the subject subsequent to the subject undergoing initiation of an assisted reproductive technique. The first hair sample or second hair sample may be collected from the subject's axilla, public area, and head.

The computer processor may be further programmed to direct the processing of the hair sample to generate a solution suspected of containing the AMH, and assay the solution for the presence of the AMH.

### Classifiers

The present disclosure provides classifiers for processing or analyzing data generated from a biological sample to yield an output. Such an output may result in an assessment of the reproductive hormone profile of a subject.

A classifier may be a machine learning algorithm. The machine learning algorithm may be a trained machine learning algorithm. The machine learning algorithm may be trained via supervised or unsupervised learning, for example. For example, the machine learning algorithm may comprise generative modeling (e.g., a statistical model of a joint probability distribution on an observable variable X on a target variable Y; such as a naïve Bayes classifier and linear discriminant analysis), discriminative modeling (e.g., a model of a conditional probability of a target variable Y, given an observation x of an observable variable X; such as a logistic regression, a perceptron, or a support vector machine), or reinforcement learning (RL).

As used herein, the terms “machine learning,” “machine learning procedure,” “machine learning operation,” and “machine learning algorithm” generally refer to any system or analytical and/or statistical procedure that may progressively (e.g., iteratively) improve computer performance of a task. Machine learning may include a machine learning algorithm. The machine learning algorithm may be a trained algorithm. Machine learning (ML) may comprise one or more supervised, semi-supervised, or unsupervised machine learning techniques. For example, an ML algorithm may be a trained algorithm that may be trained through supervised learning (e.g., various parameters are determined as weights or scaling factors). ML may comprise one or more of regression analysis, regularization, classification, dimensionality reduction, ensemble learning, meta learning, association rule learning, cluster analysis, anomaly detection, deep learning, or ultra-deep learning. ML may comprise, but may be not limited to: k-means, k-means clustering, k-nearest neighbors, learning vector quantization, linear regression, non-linear regression, least squares regression, partial least squares regression, logistic regression, stepwise regression, multivariate adaptive regression splines, ridge regression, principle component regression, least absolute shrinkage and selection operation, least angle regression, canonical correlation analysis, factor analysis, independent component analysis, linear discriminant analysis, multidimensional scaling, non-negative matrix factorization, principal components analysis, principal coordinates analysis, projection pursuit, Sammon mapping, t-distributed stochastic neighbor embedding, AdaBoosting, boosting, gradient boosting, bootstrap aggregation, ensemble averaging, decision trees, conditional decision trees, boosted decision trees, gradient boosted decision trees, random forests, stacked generalization, Bayesian networks, Bayesian belief networks, naïve Bayes, Gaussian naïve Bayes, multinomial naïve Bayes, hidden Markov models, hierarchical hidden Markov models, support vector machines, encoders, decoders, auto-encoders, stacked auto-encoders, perceptrons, multi-layer perceptrons, artificial neural networks, feedforward neural networks, convolutional neural networks, recurrent neural networks, long short-term memory, deep belief networks, deep Boltzmann machines, deep convolutional neural networks, deep recurrent neural networks, or generative adversarial networks.

As used herein, the terms “reinforcement learning,” “reinforcement learning procedure,” “reinforcement learning operation,” and “reinforcement learning algorithm” generally refer to any system or computational procedure that may take one or more actions to enhance or maximize some notion of a cumulative reward to its interaction with an environment. The agent performing the reinforcement learning (RL) procedure may receive positive or negative reinforcements, called an “instantaneous reward”, from taking one or more actions in the environment and therefore placing itself and the environment in various new states.

A goal of the agent may be to enhance or maximize some notion of cumulative reward. For instance, the goal of the agent may be to enhance or maximize a “discounted reward function” or an “average reward function”. A “Q-function” may represent the maximum cumulative reward obtainable from a state and an action taken at that state. A “value function” and a “generalized advantage estimator” may represent the maximum cumulative reward obtainable from a state given an optimal or best choice of actions. RL may utilize any one of more of such notions of cumulative reward. As used herein, any such function may be referred to as a “cumulative reward function”. Therefore, computing a best or optimal cumulative reward function may be equivalent to finding a best or optimal policy for the agent.

The agent and its interaction with the environment may be formulated as one or more Markov Decision Processes (MDPs), for example. The RL procedure may not assume knowledge of an exact mathematical model of the MDPs. The MDPs may be completely unknown, partially known, or completely known to the agent. The RL procedure may sit in a spectrum between the two extents of “model-based” or “model-free” with respect to prior knowledge of the MDPs. As such, the RL procedure may target large MDPs where exact methods may be infeasible or unavailable due to an unknown or stochastic nature of the MDPs.

The RL procedure may be implemented using one or more computer processors described herein. The digital processing unit may utilize an agent that trains, stores, and later on deploys a “policy” to enhance or maximize the cumulative reward. The policy may be sought (for instance, searched for) for a period of time that may be as long as possible or desired. Such an optimization problem may be solved by storing an approximation of an optimal policy, by storing an approximation of the cumulative reward function, or both. In some cases, RL procedures may store one or more tables of approximate values for such functions. In other cases, RL procedure may utilize one or more “function approximators”.

Examples of function approximators may include neural networks (such as deep neural networks) and probabilistic graphical models (e.g., Boltzmann machines, Helmholtz machines, and Hopfield networks). A function approximator may create a parameterization of an approximation of the cumulative reward function. Optimization of the function approximator with respect to its parameterization may consist of perturbing the parameters in a direction that enhances or maximizes the cumulative rewards and therefore enhances or optimizes the policy (such as in a policy gradient method), or by perturbing the function approximator to get closer to satisfy Bellman's optimality criteria (such as in a temporal difference method).

During training, the agent may take actions in the environment to obtain more information about the environment and about good or best choices of policies for survival or better utility. The actions of the agent may be randomly generated (for instance, especially in early stages of training) or may be prescribed by another machine learning paradigm (such as supervised learning, imitation learning, or any other machine learning procedure described herein). The actions of the agent may be refined by selecting actions closer to the agent's perception of what an enhanced or optimal policy is. Various training strategies may sit in a spectrum between the two extents of off-policy and on-policy methods with respect to choices between exploration and exploitation.

The trained algorithm may be configured to accept a plurality of input variables and to produce one or more output values based on the plurality of input variables. The plurality of input variables may comprise a presence or abundance of at least one of a reproductive hormone (e.g., dehydroepiandrosterone, estradiol, progesterone, testosterone, anti-mullerian hormone, prolactin, and vitamin D), another hormone (e.g., dehydroepiandrosterone, estradiol, progesterone, testosterone, and cortisol), and a micronutrient (e.g., folic acid, vitamin B12, lithium, vitamin B1, vitamin B2, vitamin B3, vitamin B5, vitamin B6, iron, iodine, phosphorus, potassium, selenium, and retinyl ester). The plurality of input variables may also include clinical health data of a subject. The one or more output values may comprise a state or condition of a subject. For example, the state or condition of the subject may include one or more of: assessment of ovarian reserve, assessment of reproductive lifespan, assessment (e.g., presence, absence, progression, regression, or risk) of ovarian dysfunction, assessment (e.g., presence, absence, progression, regression, or risk) of metabolic syndromes, assessment (e.g., presence, absence, progression, regression, or risk) of vitamin D deficiency, assessment (e.g., presence, absence, progression, regression, or risk) of thyroid dysfunction, and a presence, deficiency, or abundance of a reproductive hormone.

The trained algorithm may comprise a classifier, such that each of the one or more output values comprises one of a fixed number of possible values (e.g., a linear classifier, a logistic regression classifier, etc.) indicating a classification of a state or condition of the subject by the classifier. The trained algorithm may comprise a binary classifier, such that each of the one or more output values comprises one of two values (e.g., {0, 1}, {positive, negative}, {present, absent}, or {high-risk, low-risk}) indicating a classification of the state or condition of the subject. The trained algorithm may be another type of classifier, such that each of the one or more output values comprises one of more than two values (e.g., {0, 1, 2}, {positive, negative, indeterminate}, {present, absent, or indeterminate}, or {high-risk, intermediate-risk, low-risk}) indicating a classification of the state or condition of the subject.

The output values may comprise descriptive labels, numerical values, or a combination thereof. Some of the output values may comprise descriptive labels. Such descriptive labels may provide an identification or indication of a state or condition of the subject, and may comprise, for example, positive, negative, present, absent, high-risk, intermediate-risk, low-risk, or indeterminate. Such descriptive labels may provide an identification of a treatment for the state or condition of the subject, and may comprise, for example, a therapeutic intervention, a duration of the therapeutic intervention, and/or a dosage of the therapeutic intervention suitable to treat the state or condition of the subject. Such descriptive labels may provide an identification of secondary clinical tests that may be appropriate to perform on the subject, and may comprise, for example, a blood test, a genetic test, or a medical imaging. As another example, such descriptive labels may provide a prognosis of the state or condition of the subject. As another example, such descriptive labels may provide a relative assessment of the state or condition of the subject. Some descriptive labels may be mapped to numerical values, for example, by mapping “positive” to 1 and “negative” to 0.

Some of the output values may comprise numerical values, such as binary, integer, or continuous values. Such binary output values may comprise, for example, {0, 1}, {positive, negative}, {present, absent}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the state or condition of the subject. Some numerical values may be mapped to descriptive labels, for example, by mapping 1 to “positive” or “present”, and 0 to “negative” or “absent”.

Some of the output values may be assigned based on one or more cutoff values. For example, a binary classification of subjects may assign an output value of “positive”, “present”, or 1 if the subject has at least a 50% probability of having the state or condition. For example, a binary classification of subjects may assign an output value of “negative”, “absent”, or 0 if the subject has less than a 50% probability of having the state or condition. In this case, a single cutoff value of 50% is used to classify subjects into one of the two possible binary output values. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, a classification of subjects may assign an output value of “positive”, “present, or 1 if the subject has a probability of having the state or condition of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of subjects may assign an output value of “positive” or 1 if the subject has a probability of having the state or condition of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classification of subjects may assign an output value of “negative”, absent, or 0 if the subject has a probability of having the state or condition of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of subjects may assign an output value of “negative” or 0 if the subject has a probability of the state or condition of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classification of subjects may assign an output value of “indeterminate” or 2 if the subject is not classified as “positive”, “negative”, “present”, “absent”, 1, or 0. In this case, a set of two cutoff values is used to classify subjects into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify subjects into one of n+1 possible output values, where n is any positive integer.

The trained algorithm may be trained with a plurality of independent training samples. Each of the independent training samples may comprise a dataset of input variables (e.g., a presence or abundance of at least one of a reproductive hormone (e.g., dehydroepiandrosterone, estradiol, progesterone, testosterone, anti-mullerian hormone, prolactin, and vitamin D), another hormone (e.g., dehydroepiandrosterone, estradiol, progesterone, testosterone, and cortisol), and a micronutrient (e.g., folic acid, vitamin B12, lithium, vitamin B1, vitamin B2, vitamin B3, vitamin B5, vitamin B6, iron, iodine, phosphorus, potassium, selenium, and retinyl ester)) collected from a subject at a given time point, and one or more known output values (e.g., a state or condition) corresponding to the subject. Independent training samples may comprise datasets of input variables and associated output values obtained or derived from a plurality of different subjects. Independent training samples may comprise datasets of input variables and associated output values obtained at a plurality of different time points from the same subject (e.g., on a regular basis such as weekly, biweekly, or monthly). Independent training samples may be associated with presence of the state or condition (e.g., training samples comprising datasets of input variables and associated output values obtained or derived from a plurality of subjects known to have the state or condition). Independent training samples may be associated with absence of the state or condition (e.g., training samples comprising datasets of input variables and associated output values obtained or derived from a plurality of subjects who are known to not have a previous diagnosis of the state or condition or who have received a negative test result for the state or condition). A plurality of different trained algorithms may be trained, such that each of the plurality of trained algorithms is trained using a different set of independent training samples (e.g., sets of independent training samples corresponding to presence or absence of different states or conditions).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise datasets of input variables associated with presence of the state or condition and/or datasets of input variables associated with absence of the state or condition. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the state or condition. In some embodiments, the dataset of input variables is independent of samples used to train the trained algorithm.

The trained algorithm may be trained with a first number of independent training samples associated with presence of the state or condition and a second number of independent training samples associated with absence of the state or condition. The first number of independent training samples associated with presence of the state or condition may be no more than the second number of independent training samples associated with absence of the state or condition. The first number of independent training samples associated with presence of the state or condition may be equal to the second number of independent training samples associated with absence of the state or condition. The first number of independent training samples associated with presence of the state or condition may be greater than the second number of independent training samples associated with absence of the state or condition.

A machine learning algorithm may be trained with a training set of samples from subjects with identified or diagnosed conditions, such as women with a reproductive disorder. The machine learning algorithm may be trained with at least about 5, 10, 20, 30, 40, 50, 100, 200, 300, 400, 500, 1000, or more samples. Once trained, the machine learning algorithm may be used to process data generated from one or more samples independent of samples from the training set to identify one or more features in the one or more samples (e.g., a reproductive hormone, an abundance or deficiency of the reproductive hormone) at an accuracy of at least about 60%, 70%, 80%, 85%, 90%, 95%, or more. The machine learning algorithm may be used to process the data to identify the one or more features at a sensitivity of at least about 60%, 70%, 80%, 85%, 90%, 95%, or more. The machine learning algorithm may be used to process the data to identify the one or more features at a specificity of at least about 60%, 70%, 80%, 85%, 90%, 95%, or more.

The trained algorithm may be configured to identify the state or condition (e.g., assessment of ovarian reserve, assessment of reproductive lifespan, assessment (e.g., presence, absence, progression, regression, or risk) of ovarian dysfunction, assessment (e.g., presence, absence, progression, regression, or risk) of metabolic syndromes, assessment (e.g., presence, absence, progression, regression, or risk) of vitamin D deficiency, assessment (e.g., presence, absence, progression, regression, or risk) of thyroid dysfunction, and a presence, deficiency, or abundance of a reproductive hormone) at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the state or condition by the trained algorithm may be calculated as the percentage of independent test samples (e.g., subjects known to have the state or condition or subjects with negative clinical test results for the state or condition) that are correctly identified or classified as having or not having the state or condition.

The trained algorithm may be configured to identify the state or condition with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the state or condition using the trained algorithm may be calculated as the percentage of datasets of input variables identified or classified as having the state or condition that correspond to subjects that truly have the state or condition.

The trained algorithm may be configured to identify the state or condition with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the state or condition using the trained algorithm may be calculated as the percentage of datasets of input variables identified or classified as not having the state or condition that correspond to subjects that truly do not have the state or condition.

The trained algorithm may be configured to identify the state or condition with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the state or condition using the trained algorithm may be calculated as the percentage of independent test samples associated with presence of the state or condition (e.g., subjects known to have the state or condition) that are correctly identified or classified as having the state or condition.

The trained algorithm may be configured to identify the state or condition with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the state or condition using the trained algorithm may be calculated as the percentage of independent test samples associated with absence of the state or condition (e.g., subjects with negative clinical test results for the state or condition) that are correctly identified or classified as not having the state or condition.

The trained algorithm may be configured to identify the state or condition with an Area Under the Receiver Operating Characteristic (AUROC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUROC may be calculated as an integral of the Receiver Operating Characteristic (ROC) curve (e.g., the area under the ROC curve) associated with the trained algorithm in classifying datasets of input variables as having or not having the state or condition.

The trained algorithm may be adjusted or tuned to improve one or more of the performance, accuracy, PPV, NPV, clinical sensitivity, clinical specificity, or AUROC of identifying the state or condition. The trained algorithm may be adjusted or tuned by adjusting parameters of the trained algorithm (e.g., a set of cutoff values used to classify a dataset of input variables as described elsewhere herein, or parameters or weights of a neural network). The trained algorithm may be adjusted or tuned continuously during the training process or after the training process has completed.

After the trained algorithm is initially trained, a subset of the inputs may be identified as most influential or most important to be included for making high-quality classifications. For example, a subset of the plurality of features (e.g., of the input variables) may be identified as most influential or most important to be included for making high-quality classifications or identifications of the state or condition. The plurality of features or a subset thereof may be ranked based on classification metrics indicative of each feature's influence or importance toward making high-quality classifications or identifications of the state or condition. Such metrics may be used to reduce, in some cases significantly, the number of input variables (e.g., predictor variables) that may be used to train the trained algorithm to a desired performance level (e.g., based on a desired minimum accuracy, PPV, NPV, clinical sensitivity, clinical specificity, AUROC, or a combination thereof). For example, if training the trained algorithm with a plurality comprising several dozen or hundreds of input variables in the trained algorithm results in an accuracy of classification of more than 99%, then training the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics.

### Examples

### Example 1: Quantitative Competitive Enzyme Immunoassay (EIA) for Detection of Smaller Dimeric Matere Fragment (SDMF) of AMH in Hair

A quantitative competitive enzyme immunoassay (EIA) is used to detect and quantify AMH in hair, and also optionally in other biological fluids (e.g., blood, plasma, serum, and saliva). For hair sampling, all samples from all parts of the body are collected using an electric hair clipper, aiming to cut the hair as close to the skin as possible. Hair harvesting is performed carefully to prevent hair follicle removal and avoid skin damage. The peeler blade is cleaned between each hair collection with a brush. Each hair sample weighs approximately 150-200 mg. All hair samples are stored into individually identified zip-lock plastic bags kept in a dark container at room temperature until hormone extraction.

For hAMH extraction, 100-150 milligrams of hair is weighed from each sample and placed into a 15-mL conical tube. Each sample is washed by adding 1-2.5 mL of isopropanol and vortexed for five seconds to wash the sample from presumptive external contamination, e.g., from sweat glands. The supernatant is separated by decantation. After washing, hair samples are left to dry completely at 30-37° C. Once dried, hair is trimmed using a ball mill for up to 15 minutes at 22 Hz (MM200, Retsch, Haan, Germany). For the steroid extraction, between 30 and 70 mg of trimmed hair is carefully weighed, placed into a 2-mL Eppendorf tube and 0.8-2 mL of pure methanol and 0.3-1.5 mL of milli-Q distilled water is added. Samples are incubated for up to 24 hours at 30-37° C. under moderate shaking. After incubation, extracted samples are centrifuged at 7 000-10000×g for 2 to 10 minutes and 0.5-1 mL of supernatant transferred into a new 2-mL Eppendorf tube. The supernatant is then placed in an oven at 38-40° C. to evaporate the extract. Once the extract is completely evaporated, the samples are reconstituted with 0.200-0.300 mL of EIA buffer and shaken for 30 seconds. Samples are then immediately stored at 20° C. until analysis.

Polyclonal or Monoclonal antibodies that specifically recognize epitopes on AMH are used as “capture antibodies.” The capture antibody is immobilized onto a solid support (e.g., ELISA plate or a cartridge). Recombinant AMH peptides or synthetic mimic peptides (e.g., 30-40 amino acids, which encompass AMH regions recognized by capture antibody) are conjugated to a detection tag, such as an enzyme (horseradish peroxidase), a fluorescent tag, radioactive tag, etc., and are used as “labeled antigens.” The sample (e.g., saliva or hair), which contains AMH (“unlabeled antigen,”) is then mixed with the “labeled antigen,” and the mixture is brought in contact with the immobilized “capture antibody.” Both the sample (unlabeled antigen) and labeled antigen bind to the capture antibody and “compete” for binding to the limited number of capture antibodies. After the mixture is allowed to bind, unbound sample and labeled antigen are washed off. Then a substrate (e.g., colorimetric/chromogenic substrate such as 3,3′, 5, 5′-tetramethylbenzidne (TMB) for HRP) is added. The reaction is then stopped. The enzyme-substrate reaction is detected and/or quantified using a detection platform (e.g., spectrophotometer). The signal obtained is inversely proportional to the amount of AMH (e.g., the protein of interest) in the sample.

### Example 2: Quantitative Competitive Enzyme Immunoassay (EIA) for Detection of Smaller Dimeric Mature Fragment (SDMF) of AMH in Biological Fluids

A quantitative competitive enzyme immunoassay (EIA) is used to detect and quantify SDMF of AMH in biological fluids (e.g., blood, plasma serum, and saliva). A quantitative competitive enzyme immunoassay (EIA) is used to detect and quantify AMH in biological fluids (e.g., blood, plasma, serum, and saliva).

For saliva sampling, samples are collected. All saliva samples are stored into individually identified zip-lock plastic bags kept in a dark container at room temperature until hormone extraction.

For AMH extraction, up to 5 mL of saliva is collected in an inert plastic tube. A part of saliva is then transferred to a different tube and evaporated. The dried saliva is then reconstituted in a proprietary buffer and ELISA is performed on this extract.

Polyclonal or Monoclonal antibodies that specifically recognize epitopes on SDMF which become conformationally exposed (or available) following dissociation of the SDMF from the pro-region (non-active), are used as “capture antibodies.” The capture antibody is immobilized onto a solid support (e.g., ELISA plate or a cartridge). Recombinant AMH peptides or synthetic mimic peptides (e.g., 30-40 amino acids, which encompass AMH regions recognized by capture antibody) are conjugated to a detection tag, such as an enzyme (horseradish peroxidase), a fluorescent tag, radioactive tag, etc., and are used as “labeled antigens.” The sample (e.g., saliva), which contains SDMF (“unlabeled antigen,”) is then mixed with the “labeled antigen,” and the mixture is brought in contact with the immobilized “capture antibody.” Both the sample (unlabeled antigen) and labeled antigen bind to the capture antibody and “compete” for binding to the limited number of capture antibodies. After the mixture is allowed to bind, unbound sample and labeled antigen are washed off. Then a substrate (e.g., colorimetric/chromogenic substrate such as 3,3′, 5, 5′-tetramethylbenzidne (TMB) for HRP) is added. The reaction is then stopped. The enzyme-substrate reaction is then detected and/or quantified using a detection platform (e.g., spectrophotometer). The signal obtained is inversely proportional to the amount of SDMF (e.g., the protein of interest) in the sample.

In a separate assay, total AMH is measured using a commercially-available EIA tests (e.g., Ansh, Beckman-Coulter). A ratio of SDMF/AMH is then computed. High ratios are considered as indicative (diagnostic) for high turnover of active AMH from the receptor and effectively low active AMH.

### Example 3: Quantitative Competitive Enzyme Immunoassay (EIA) for Detection of AMH in Hair

A quantitative competitive enzyme immunoassay (EIA) was used to detect and quantify AMH in hair, and also optionally in other biological fluids (e.g., blood, serum, and saliva). For hair sampling, all samples from all parts of the body were collected using an electric hair clipper, aiming to cut the hair as close to the skin as possible. Hair harvesting was performed carefully to prevent hair follicle removal and avoid skin damage. The peeler blade was cleaned between each hair collection with a brush. Each hair sample weighed approximately 150-200 mg. All hair samples were stored into individually identified zip-lock plastic bags kept in a dark container at room temperature until hormone extraction.

For hAMH extraction, 100-150 milligrams of hair was weighed from each sample and placed into a 15-mL conical tube. Assay conditions were developed by evaluating different extraction buffers with different proportions thereof, previous wash with hydrogen peroxide, intensity of the ball mill parameters, etc. For example, extraction buffer such as ultrapure water, EIA buffer solution, methanol, and ethanol with different proportions, such as 50%, 80%, and 100% were evaluated. Each sample was washed by adding 1-2.5 mL of isopropanol and vortexed for five seconds to wash the sample from presumptive external contamination, e.g., from sweat glands. The supernatant was separated by decantation. After washing, hair samples were left to dry completely at 30-37° C. Once dried, hair was trimmed using a ball mill for up to 15 minutes at 22 Hz (MM200, Retsch, Haan, Germany). For the steroid extraction, between 30 and 70 mg of trimmed hair was carefully weighed, placed into a 2-mL Eppendorf tube, and 0.8-2 mL of pure methanol and 0.3-1.5 mL of milli-Q distilled water were added. Samples were incubated for up to 24 hours at 30-37° C. under moderate shaking. After incubation, extracted samples were centrifuged at 7 000-10000×g for 2 to 10 minutes and 0.5-1 mL of supernatant was transferred into a new 2-mL Eppendorf tube. The supernatant was then placed in an oven at 38-40° C. to evaporate the extract. Once the extract was completely evaporated, the samples were reconstituted with 0.200-0.300 mL of EIA buffer and shaken for 30 seconds. Samples were then immediately stored at 20° C. until analysis.

Polyclonal or Monoclonal antibodies that specifically recognize epitopes on AMH were used as “capture antibodies.” The capture antibody was immobilized onto a solid support (e.g., ELISA plate or a cartridge). Recombinant AMH peptides or synthetic mimic peptides (e.g., 30-40 amino acids, which encompass AMH regions recognized by capture antibody) were conjugated to a detection tag, such as an enzyme (horseradish peroxidase), a fluorescent tag, radioactive tag, etc., and are used as “labeled antigens.” The sample (e.g., saliva or hair), which contains AMH (“unlabeled antigen,”) was then mixed with the “labeled antigen,” and the mixture was brought in contact with the immobilized “capture antibody.” Both the sample (unlabeled antigen) and labeled antigen bind to the capture antibody and “compete” for binding to the limited number of capture antibodies. After the mixture was allowed to bind, the unbound sample and labeled antigen were washed off. Then a substrate (e.g., colorimetric/chromogenic substrate such as 3,3′, 5, 5′-tetramethylbenzidne (TMB) for HRP) was added. The reaction was then stopped. The enzyme-substrate reaction was detected and/or quantified using a detection platform (e.g., spectrophotometer). The signal obtained was inversely proportional to the amount of AMH (e.g., the protein of interest) in the sample.

TABLE 1
Levels of AMH detected
in hair samples

| | AMH |
|---|---|
| Sample | levels |
| ID | (ng/mg) |
| 1 | 0.011 |
| 2 | 0.019 |
| 3 | 0.023 |
| 4 | 0.015 |
| 5 | 0.037 |
| 6 | 0.014 |
| 7 | 0.029 |
| 8 | 0.012 |
| 9 | 0.017 |
| 10 | 0.039 |
| 11 | 0.032 |
| 12 | 0.016 |
| 13 | 0.022 |
| 14 | 0.028 |
| 15 | 0.045 |
| 16 | 0.038 |
| 17 | 0.018 |
| 18 | 0.023 |
| 19 | 0.045 |
| 20 | 0.033 |
| 21 | 0.050 |
| 22 | 0.018 |
| 23 | 0.014 |
| 24 | 0.012 |
| 25 | 0.034 |
| 26 | 0.048 |
| 27 | 0.028 |
| 28 | 0.012 |
| 29 | 0.014 |
| 30 | 0.033 |
| 31 | 0.048 |
| 32 | 0.053 |
| 33 | 0.015 |
| 34 | 0.018 |
| 35 | 0.028 |
| 36 | 0.037 |
| 37 | 0.043 |
| 38 | 0.032 |
| 39 | 0.019 |
| 40 | 0.018 |
| 41 | 0.023 |
| 42 | 0.034 |
| 43 | 0.038 |
| 44 | 0.018 |
| 45 | 0.019 |
| 46 | 0.046 |
| 47 | 0.038 |
| 48 | 0.035 |
| 49 | 0.033 |
| 50 | 0.017 |
| 51 | 0.019 |
| 52 | 0.048 |
| 53 | 0.054 |
| 54 | 0.025 |
| 55 | 0.043 |
| 56 | 0.035 |
| 57 | 0.049 |
| 58 | 0.032 |
| 59 | 0.027 |
| 60 | 0.018 |
| 61 | 0.019 |
| 62 | 0.021 |
| 63 | 0.055 |
| 64 | 0.023 |
| 65 | 0.045 |
| 66 | 0.056 |
| 67 | 0.023 |
| 68 | 0.018 |
| 69 | 0.033 |
| 70 | 0.026 |
| 71 | 0.018 |
| 72 | 0.029 |
| 73 | 0.034 |
| 74 | 0.059 |
| 75 | 0.055 |
| 76 | 0.045 |
| 77 | 0.036 |
| 78 | 0.019 |
| 79 | 0.018 |
| 80 | 0.025 |
| 81 | 0.057 |
| 82 | 0.043 |
| 83 | 0.039 |
| 84 | 0.036 |
| 85 | 0.034 |
| 86 | 0.019 |
| 87 | 0.032 |
| 88 | 0.020 |
| 89 | 0.060 |
| 90 | 0.056 |
| 91 | 0.035 |
| 92 | 0.021 |
| 93 | 0.036 |
| 94 | 0.045 |
| 95 | 0.056 |
| 96 | 0.035 |
| 97 | 0.029 |
| 98 | 0.043 |
| 99 | 0.026 |
| 100 | 0.019 |
| 101 | 0.021 |
| 102 | 0.017 |
| 103 | 0.028 |
| 104 | 0.027 |
| 105 | 0.024 |
| 106 | 0.017 |
| 107 | 0.029 |
| 108 | 0.028 |
| 109 | 0.021 |
| 110 | 0.012 |

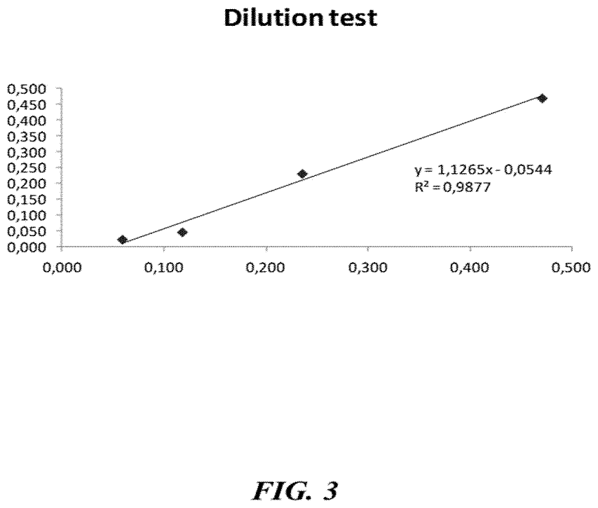

As illustrated in Table 1, AMH levels were detected and measured in collected hair samples. Thus, methods of the present disclosure may be applied for determining AMH levels in hair. AMH levels may accurately reflect the size of the antral follicles pool, which represents the quantity of remaining primordial follicles. Determining AMH may also be useful in predicting the start of menopause, and to help diagnose polycystic ovary syndrome. As shown in FIG. 3, a linearity or dilution test provides confirmation that AMH in hair extracts interacted with the assay antibody in a dose-dependent manner. Further, the dilution test supports the assumption that the antibody-binding characteristics of standard AMH from the assay and AMH from hair samples are similar. The specification of the dilution test is high, as demonstrated by the high coefficient of determination (R²) of at 98.77%. Other assays of validation also support the above assumption. For example, the precision of dilution test is accurate, as demonstrated by coefficient of variation of intra-assay at 8.04% and coefficient of variation of inter-assay at 12.52%.

### Example 4: Quantitative Competitive Enzyme Immunoassay (EIA) for Detection of AMH and Other Hormones in Hair and in Plasma

A quantitative EIA was used to detect and quantify AMH and other hormones in hair samples and plasma samples collected from the same individual.

TABLE 2
Levels of AMH and other hormones detected
in hair and plasma samples

| | Hair | | Plasma | |
|---|---|---|---|---|
| Sample | Progesterone | AMH | Progesterone | AMH |
| ID | (ng/mg) | (pg/mg) | (ng/ml) | (ng/ml) |
| 1 | 0.81 | 3.32 | 0.14 | 8.46 |
| 2 | 2.83 | 2.95 | 0.22 | 3.66 |
| 3 | 1.27 | 2.86 | 0.94 | 3.12 |
| 4 | 1.44 | 1.87 | 0.29 | 0.9 |
| 5 | 1.44 | 2.75 | 0.35 | 2.89 |

Table 2 shows that AMH was detected in hair. Methods of the present disclosure may be applied for determining AMH levels in hair.

Further, Table 3 shows that cortisol levels were detected in hair samples. Methods of the present disclosure may be used for isolating cortisol and determining cortisol levels in hair samples. Cortisol levels may also be optionally detected in in biological fluids (e.g., blood, plasma serum, and saliva).

TABLE 3
Levels of Cortisol detected
in hair samples

| Sample | Cortisol |
|---|---|
| ID | (pg/mg) |
| 1 | 7.88 |
| 2 | 3.86 |
| 3 | 1.72 |
| 4 | 5.37 |
| 5 | 1.03 |
| 6 | 1.66 |
| 7 | 13.03 |
| 8 | 1.34 |
| 9 | 1.62 |
| 10 | 6.82 |
| 11 | 3.38 |
| 12 | 2.01 |
| 13 | 0.77 |
| 14 | 9.42 |
| 15 | 10.29 |
| 16 | 1.25 |
| 17 | 3.72 |
| 18 | 0.31 |
| 19 | 1.18 |
| 20 | 1.88 |
| 21 | 13.59 |
| 22 | 0.96 |
| 23 | 0.62 |
| 24 | 2.42 |
| 25 | 7.85 |
| 26 | 6.90 |
| 27 | 3.24 |
| 28 | 2.39 |
| 29 | 1.69 |
| 30 | 2.50 |
| 31 | 0.34 |
| 32 | 3.54 |
| 33 | 2.99 |
| 34 | 4.08 |
| 35 | 6.62 |
| 36 | 33.51 |
| 37 | 4.14 |
| 38 | 1.67 |
| 39 | 9.73 |
| 40 | 2.03 |
| 41 | 2.03 |
| 42 | 5.18 |
| 43 | 2.32 |
| 44 | 0.94 |
| 45 | 7.79 |
| 46 | 2.54 |
| 47 | 8.11 |
| 48 | 5.85 |
| 49 | 5.25 |
| 50 | 4.28 |
| 51 | 19.57 |
| 52 | 1.23 |
| 53 | 1.82 |
| 54 | 3.57 |
| 55 | 3.25 |
| 56 | 2.28 |
| 57 | 6.57 |
| 58 | 0.46 |
| 59 | 7.20 |
| 60 | 2.56 |
| 61 | 3.31 |
| 62 | 6.60 |
| 63 | 3.08 |
| 64 | 1.87 |
| 65 | 2.55 |
| 66 | 12.32 |
| 67 | 3.86 |
| 68 | 5.41 |
| 69 | 4.76 |
| 70 | 8.70 |
| 71 | 6.20 |
| 72 | 6.66 |
| 73 | 1.82 |
| 74 | 1.69 |
| 75 | 2.58 |
| 76 | 3.09 |

### Example 5: Quantitative Competitive Enzyme Immunoassay (EIA) for Detection of DHEA or DHEA-S in Hair

A quantitative competitive enzyme immunoassay (EIA) was used to detect and quantify DHEA or DHEA-S in hair, and also in other biological fluids (e.g., blood, serum, and saliva). For hair sampling, all samples from all parts of the body were collected using an electric hair clipper, aiming to cut the hair as close to the skin as possible. Hair harvesting was performed carefully to prevent hair follicle removal and avoid skin damage. The peeler blade was cleaned between each hair collection with a brush. Each hair sample weighed approximately 150-200 mg. All hair samples were stored into individually identified zip-lock plastic bags kept in a dark container at room temperature until hormone extraction.

For DHEA/DHEA-S extraction, 100-150 milligrams of hair was weighed from each sample and placed into a 15-mL conical tube. Each sample was washed by adding 1-2.5 mL of isopropanol and vortexed for five seconds to wash the sample from presumptive external contamination, e.g., from sweat glands. The supernatant was separated by decantation. After washing, hair samples were left to dry completely at 30-37° C. Once dried, hair was trimmed using a ball mill for up to 15 minutes at 22 Hz (MM200, Retsch, Haan, Germany). For the steroid extraction, between 30 and 70 mg of trimmed hair was carefully weighed, placed into a 2-mL Eppendorf tube, and 0.8-2 mL of pure methanol and 0.3-1.5 mL of milli-Q distilled water were added. Samples were incubated for up to 24 hours at 30-37° C. under moderate shaking. After incubation, extracted samples were centrifuged at 7 000-10000×g for 2 to 10 minutes and 0.5-1 mL of supernatant was transferred into a new 2-mL Eppendorf tube. The supernatant was then placed in an oven at 38-40° C. to evaporate the extract. Once the extract was completely evaporated, the samples were reconstituted with 0.200-0.300 mL of EIA buffer and shaken for 30 seconds. Samples were then immediately stored at 20° C. until analysis.

Polyclonal or Monoclonal antibodies that specifically recognize epitopes on DHEA/DHEA-S were used as “capture antibodies.” The capture antibody was immobilized onto a solid support (e.g., ELISA plate or a cartridge). Recombinant DHEA/DHEA-S peptides or synthetic mimic peptides (e.g., 30-40 amino acids, which encompass DHEA regions recognized by capture antibody) were conjugated to a detection tag, such as an enzyme (horseradish peroxidase), a fluorescent tag, radioactive tag, etc., and are used as “labeled antigens.” The sample (e.g., saliva or hair), which contains DHEA (“unlabeled antigen,”) was then mixed with the “labeled antigen,” and the mixture was brought in contact with the immobilized “capture antibody.” Both the sample (unlabeled antigen) and labeled antigen bind to the capture antibody and “compete” for binding to the limited number of capture antibodies. After the mixture was allowed to bind, unbound sample and labeled antigen were washed off. Then a substrate (e.g., colorimetric/chromogenic substrate such as 3,3′, 5, 5′-tetramethylbenzidne (TMB) for HRP) was added. The reaction was then stopped. The enzyme-substrate reaction was detected and/or quantified using a detection platform (e.g., spectrophotometer). The signal obtained was inversely proportional to the amount of AMH (e.g., the protein of interest) in the sample.

TABLE 4
Levels of DHEA detected in hair samples

| Sample | | DHEA levels |
|---|---|---|
| ID | Sex | (pg/mg) |
| 1 | | 96.999 |
| 2 | | 195.499 |
| 3 | Male | 303.999 |
| 4 | | 303.999 |
| 5 | | 172.399 |
| 6 | Female | 172.399 |

Table 4 shows that DHEA was detected in hair. Thus, methods of the present disclosure may be applied for determining DHEA levels in hair. For example, the measurement of DHEA/DHEA-S concentrations in hair may make it possible to assess long-term adrenal or gonadal activity without the need of serial and continuous sampling. Determining DHEA/DHEA-S may be useful in evaluating adrenal gland function and helping diagnose polycystic ovary syndrome.

### Example 6: Quantitative Competitive Enzyme Immunoassay (EIA) for Detection of Estradiol in Hair

A quantitative competitive enzyme immunoassay (EIA) was used to detect and quantify estradiol in hair, and also in other biological fluids (e.g., blood, serum, and saliva). For hair sampling, all samples from all parts of the body were collected using an electric hair clipper, aiming to cut the hair as close to the skin as possible. Hair harvesting was performed carefully to prevent hair follicle removal and avoid skin damage. The peeler blade was cleaned between each hair collection with a brush. Each hair sample weighed approximately 150-200 mg. All hair samples were stored into individually identified zip-lock plastic bags kept in a dark container at room temperature until hormone extraction.

For estradiol extraction, 100-150 milligrams of hair was weighed from each sample and placed into a 15-mL conical tube. Each sample was washed by adding 1-2.5 mL of isopropanol and vortexed for five seconds to wash the sample from presumptive external contamination, e.g., from sweat glands. The supernatant was separated by decantation. After washing, hair samples were left to dry completely at 30-37° C. Once dried, hair was trimmed using a ball mill for up to 15 minutes at 22 Hz (MM200, Retsch, Haan, Germany). For the steroid extraction, between 30 and 70 mg of trimmed hair was carefully weighed, placed into a 2-mL Eppendorf tube, and 0.8-2 mL of pure methanol and 0.3-1.5 mL of milli-Q distilled water were added. Samples were incubated for up to 24 hours at 30-37° C. under moderate shaking. After incubation, extracted samples were centrifuged at 7 000-10000×g for 2 to 10 minutes and 0.5-1 mL of supernatant was transferred into a new 2-mL Eppendorf tube. The tube was then placed in an oven at 38-40° C. to evaporate the extract. Once the extract was completely evaporated, the samples were reconstituted with 0.200-0.300 mL of EIA buffer and shaken for 30 seconds. Samples were then immediately stored at 20° C. until analysis.

Polyclonal or Monoclonal antibodies that specifically recognize epitopes on estradiol were used as “capture antibodies.” The capture antibody was immobilized onto a solid support (e.g., ELISA plate or a cartridge). Recombinant estradiol peptides or synthetic mimic peptides (e.g., 30-40 amino acids, which encompass estradiol regions recognized by capture antibody) were conjugated to a detection tag, such as an enzyme (horseradish peroxidase), a fluorescent tag, radioactive tag, etc., and are used as “labeled antigens.” The sample (e.g., saliva or hair), which contains AMH (“unlabeled antigen,”) was then mixed with the “labeled antigen,” and the mixture is brought in contact with the immobilized “capture antibody.” Both the sample (unlabeled antigen) and labeled antigen bind to the capture antibody and “compete” for binding to the limited number of capture antibodies. After the mixture was allowed to bind, the unbound sample and labeled antigen were washed off. Then a substrate (e.g., colorimetric/chromogenic substrate such as 3,3′, 5, 5′-tetramethylbenzidne (TMB) for HRP) was added. The reaction was then stopped. The enzyme-substrate reaction was detected and/or quantified using a detection platform (e.g., spectrophotometer). The signal obtained was inversely proportional to the amount of AMH (e.g., the protein of interest) in the sample.

TABLE 5
Levels of estradiol detected in hair samples

| Sample | | Estradiol levels |
|---|---|---|
| ID | Sex | (pg/mg) |
| 1 | Female | 3.79 |
| 2 | | 4.071 |
| 3 | | 3.882 |
| 4 | | 7.171 |
| 5 | | 0.882 |
| 6 | Female | 6.299 |
| 7 | Female | 5.519 |
| 8 | Female | 5.179 |
| 9 | Female | 2.93 |
| 10 | Female | 4.97 |
| 11 | Female | 8.72 |
| 12 | Male | 5.93 |
| 13 | Female | 18.42 |
| 14 | Female | 12.64 |
| 15 | Female | 16.12 |
| 16 | Female | 13.06 |
| 17 | Female | 24.59 |
| 18 | Female | 17.21 |
| 19 | Female | 20.42 |
| 20 | Female | 29.59 |
| 21 | Female | 19.92 |
| 22 | Female | 17.75 |
| 23 | Female | 10.34 |
| 24 | Female | 10.61 |
| 25 | Female | 28.03 |
| 26 | Female | 19.81 |
| 27 | Female | 7.223 |
| 28 | Female | 6.903 |

Table 5 shows that estradiol was detected in hair. Thus, methods of the present disclosure may be applied for determining estradiol levels in hair. For example, determining estradiol may be useful in assessing fertility, predicting the start of menopause, and to help diagnose polycystic ovary syndrome.

### Example 7: Quantitative Competitive Enzyme Immunoassay (EIA) for Detection of Progesterone in Hair

A quantitative competitive enzyme immunoassay (EIA) was used to detect and quantify progesterone in hair, and also in other biological fluids (e.g., blood, serum, saliva). For hair sampling, all samples from all parts of the body were collected using an electric hair clipper, aiming to cut the hair as close to the skin as possible. Hair harvesting was performed carefully to prevent hair follicle removal and avoid skin damage. The peeler blade was cleaned between each hair collection with a brush. Each hair sample weighed approximately 150-200 mg. All hair samples were stored into individually identified zip-lock plastic bags kept in a dark container at room temperature until hormone extraction.

For progesterone extraction, 100-150 milligrams of hair was weighed from each sample and placed into a 15-mL conical tube. Each sample was washed by adding 1-2.5 mL of isopropanol and vortexed for five seconds to wash the sample from presumptive external contamination, e.g., from sweat glands. The supernatant was separated by decantation. After washing, hair samples were left to dry completely at 30-37° C. Once dried, hair was trimmed using a ball mill for up to 15 minutes at 22 Hz (MM200, Retsch, Haan, Germany). For the steroid extraction, between 30 and 70 mg of trimmed hair was carefully weighed, placed into a 2-mL Eppendorf tube, and 0.8-2 mL of pure methanol and 0.3-1.5 mL of milli-Q distilled water were added. Samples were incubated for up to 24 hours at 30-37° C. under moderate shaking. After incubation, extracted samples were centrifuged at 7 000-10000×g for 2 to 10 minutes and 0.5-1 mL of supernatant was transferred into a new 2-mL Eppendorf tube. The supernatant was then placed in an oven at 38-40° C. to evaporate the extract. Once the extract was completely evaporated, the samples were reconstituted with 0.200-0.300 mL of EIA buffer and shaken for 30 seconds. Samples were then immediately stored at 20° C. until analysis.

Polyclonal or Monoclonal antibodies that specifically recognize epitopes on progesterone were used as “capture antibodies.” The capture antibody was immobilized onto a solid support (e.g., ELISA plate or a cartridge). Recombinant progesterone peptides or synthetic mimic peptides (e.g., 30-40 amino acids, which encompass progesterone regions recognized by capture antibody) were conjugated to a detection tag, such as an enzyme (horseradish peroxidase), a fluorescent tag, radioactive tag, etc., and were used as “labeled antigens.” The sample (e.g., saliva or hair), which contains progesterone (“unlabeled antigen,”) was then mixed with the “labeled antigen,” and the mixture was brought in contact with the immobilized “capture antibody.” Both the sample (unlabeled antigen) and labeled antigen bind to the capture antibody and “compete” for binding to the limited number of capture antibodies. After the mixture was allowed to bind, unbound sample and labeled antigen were washed off. Then a substrate (e.g., colorimetric/chromogenic substrate such as 3,3′, 5, 5′-tetramethylbenzidne (TMB) for HRP) was added. The reaction was then stopped. The enzyme-substrate reaction was detected and/or quantified using a detection platform (e.g., spectrophotometer). The signal obtained was inversely proportional to the amount of progesterone (e.g., the protein of interest) in the sample.

TABLE 6
Levels of progesterone detected in hair samples

| | | Progesterone | | | |
|---|---|---|---|---|---|
| Sample | | levels | Sample | | Progesterone |
| ID | Sex | (pg/mg) | ID | Sex | (pg/mg) |
| 1 | Female | 255.829 | 40 | Female | 225.212 |
| 2 | Female | 362.805 | 41 | Female | 179.372 |
| 3 | Female | 80.215 | 42 | Female | 186.506 |
| 4 | Female | 101.6 | 43 | Female | 225.495 |
| 5 | Female | 261.224 | 44 | Female | 433.41 |
| 6 | Female | 213.301 | 45 | Female | 370.485 |
| 7 | Female | 334.434 | 46 | Female | 361.243 |
| 8 | Female | 325.15 | 47 | Female | 268.086 |
| 9 | Female | 193.31 | 48 | Female | 477.211 |
| 10 | Female | 137.534 | 49 | Female | 88.494 |
| 11 | Female | 100.501 | 50 | Female | 1513.367 |
| 12 | Female | 210.102 | 51 | Female | 131.891 |
| 13 | | 146.714 | 52 | Female | 124.241 |
| 14 | | 156.201 | 53 | Female | 246.023 |
| 15 | | 160.718 | 54 | Female | 163.992 |
| 16 | Female | 125.573 | 55 | Female | 347.264 |
| 17 | | 181.366 | 56 | Female | 349.967 |
| 18 | Female | 358.673 | 57 | Female | 208.949 |
| 19 | Female | 281.515 | 58 | Female | 87.908 |
| 20 | Female | 179.072 | 59 | Female | 251.318 |
| 21 | Female | 146.78 | 60 | Female | 177.422 |
| 22 | Female | 197.89 | 61 | Female | 131.227 |
| 23 | Female | 280.23 | 62 | Female | 443.122 |
| 24 | Male | 191.99 | 63 | Female | 286.035 |
| 25 | Female | 294.753 | 64 | Female | 256.152 |
| 26 | Female | 253.133 | 65 | Female | 244.921 |
| 27 | Female | 224.678 | 66 | Female | 189.025 |
| 28 | Female | 118.089 | 67 | Female | 394.325 |
| 29 | Female | 206.748 | 68 | Female | 246.144 |
| 30 | Female | 385.139 | 69 | Female | 336.895 |
| 31 | Female | 170.387 | 70 | Female | 457.459 |
| 32 | Female | 157.474 | 71 | Female | 385.568 |
| 33 | Female | 194.469 | 72 | Female | 273.29 |
| 34 | Female | 207.372 | 73 | Female | 188.086 |
| 35 | Female | 260.988 | 74 | Female | 171.954 |
| 36 | Female | 233.385 | 75 | Female | 488.386 |
| 37 | Female | 118.283 | 76 | Female | 228.421 |
| 38 | Female | 125.913 | 78 | Female | 178.946 |
| 39 | Female | 262.463 | 79 | Female | 215.166 |

Table 6 shows that progesterone was detected in hair. Thus, methods of the present disclosure may be applied for determining progesterone levels in hair. For example, determining progesterone may be useful in assessing fertility.

### Example 8: Quantitative Competitive Enzyme Immunoassay (EIA) for Detection of Testosterone in Hair

A quantitative competitive enzyme immunoassay (EIA) was used to detect and quantify testosterone in hair, and also optionally in other biological fluids (e.g., blood, serum, and saliva). For hair sampling, all samples from all parts of the body were collected using an electric hair clipper, aiming to cut the hair as close to the skin as possible. Hair harvesting was performed carefully to prevent hair follicle removal and avoid skin damage. The peeler blade was cleaned between each hair collection with a brush. Each hair sample weighed approximately 150-200 mg. All hair samples were stored into individually identified zip-lock plastic bags kept in a dark container at room temperature until hormone extraction.

For testosterone extraction, 100-150 milligrams of hair was weighed from each sample and placed into a 15-mL conical tube. Each sample was washed by adding 1-2.5 mL of isopropanol and vortexed for five seconds to wash the sample from presumptive external contamination, e.g., from sweat glands. The supernatant was separated by decantation. After washing, hair samples were left to dry completely at 30-37° C. Once dried, hair was trimmed using a ball mill for up to 15 minutes at 22 Hz (MM200, Retsch, Haan, Germany). For the steroid extraction, between 30 and 70 mg of trimmed hair was carefully weighed, placed into a 2-mL Eppendorf tube, and 0.8-2 mL of pure methanol and 0.3-1.5 mL of milli-Q distilled water were added. Samples were incubated for up to 24 hours at 30-37° C. under moderate shaking. After incubation, extracted samples were centrifuged at 7,000-10000×g for 2 to 10 minutes, and 0.5-1 mL of supernatant was transferred into a new 2-mL Eppendorf tube. The supernatant was then placed in an oven at 38-40° C. to evaporate the extract. Once the extract was completely evaporated, the samples were reconstituted with 0.200-0.300 mL of EIA buffer and shaken for 30 seconds. Samples were then immediately stored at 20° C. until analysis.

Polyclonal or Monoclonal antibodies that specifically recognize epitopes on testosterone were used as “capture antibodies.” The capture antibody was immobilized onto a solid support (e.g., ELISA plate or a cartridge). Recombinant testosterone peptides or synthetic mimic peptides (e.g., 30-40 amino acids, which encompass testosterone regions recognized by capture antibody) were conjugated to a detection tag, such as an enzyme (horseradish peroxidase), a fluorescent tag, radioactive tag, etc., and are used as “labeled antigens.” The sample (e.g., saliva or hair), which contains testosterone (“unlabeled antigen,”) was then mixed with the “labeled antigen,” and the mixture was brought in contact with the immobilized “capture antibody.” Both the sample (unlabeled antigen) and labeled antigen bind to the capture antibody and “compete” for binding to the limited number of capture antibodies. After the mixture was allowed to bind, unbound sample and labeled antigen were washed off. Then a substrate (e.g., colorimetric/chromogenic substrate such as 3,3′, 5, 5′-tetramethylbenzidne (TMB) for HRP) was added. The reaction was then stopped. The enzyme-substrate reaction was detected and/or quantified using a detection platform (e.g., spectrophotometer). The signal obtained was inversely proportional to the amount of testosterone (e.g., the protein of interest) in the sample.

TABLE 7
Levels of testosterone detected in hair samples

| | | Testosterone |
|---|---|---|
| Sample | | levels |
| ID | Sex | (pg/mg) |
| 1 | Male | 3.817 |
| 2 | Male | 3.221 |
| 3 | Male | 18.635 |
| 4 | Male | 13.609 |
| 5 | Male | 48.114 |
| 6 | Male | 6.315 |
| 7 | Male | 14.367 |
| 8 | Male | 18.896 |
| 9 | Male | 15.947 |
| 10 | Male | 8.271 |
| 11 | Male | 5.713 |
| 12 | Male | 8.067 |
| 13 | Male | 6.493 |
| 14 | Male | 7.258 |
| 15 | Male | 6.865 |
| 16 | Male | 17.676 |
| 17 | Male | 7.237 |
| 18 | Male | 5.249 |
| 19 | Male | 12.502 |
| 20 | Male | 8.18 |
| 21 | Male | 3.946 |
| 22 | Male | 7.298 |
| 23 | Male | 10.803 |
| 24 | Male | 17.143 |
| 25 | Male | 24.68 |
| 26 | Male | 44.262 |
| 27 | Male | 13.609 |
| 28 | Male | 16.58 |
| 29 | Male | 11.678 |
| 30 | Male | 12.108 |
| 31 | Male | 13.383 |
| 32 | Male | 11.711 |
| 33 | Male | 18.326 |
| 34 | Male | 17.48 |
| 35 | Male | 7.694 |
| 36 | Male | 7.277 |
| 37 | Male | 20.391 |
| 38 | Female | 3.853 |
| 39 | Female | 4.723 |
| 40 | Female | 4.629 |
| 41 | Female | 4.643 |
| 42 | Female | 4.669 |
| 43 | Female | 4.669 |
| 44 | Female | 4.669 |
| 45 | Female | 4.819 |
| 46 | Female | 4.511 |
| 47 | Female | 4.669 |
| 48 | Female | 4.537 |

Table 7 shows that testosterone may be detected in hair. Thus, methods of the present disclosure may be applied for determining testosterone levels in hair. For example, determining testosterone may be useful in assessing fertility, and to help diagnose polycystic ovary syndrome.

### Example 9: Quantitative Competitive Enzyme Immunoassay (EIA) for Detection of T3 in Hair

A quantitative competitive enzyme immunoassay (EIA) was used to detect and quantify T3 in hair, and also optionally in other biological fluids (e.g., blood, serum, and saliva). For hair sampling, all samples from all parts of the body were collected using an electric hair clipper, aiming to cut the hair as close to the skin as possible. Hair harvesting was performed carefully to prevent hair follicle removal and avoid skin damage. The peeler blade was cleaned between each hair collection with a brush. Each hair sample weighed approximately 150-200 mg. All hair samples were stored into individually identified zip-lock plastic bags kept in a dark container at room temperature until hormone extraction.

For T3 extraction, 100-150 milligrams of hair was weighed from each sample and placed into a 15-mL conical tube. Each sample was washed by adding 1-2.5 mL of isopropanol and vortexed for five seconds to wash the sample from presumptive external contamination, e.g., from sweat glands. The supernatant was separated by decantation. After washing, hair samples were left to dry completely at 30-37° C. Once dried, hair was trimmed using a ball mill for up to 15 minutes at 22 Hz (MM200, Retsch, Haan, Germany). For the steroid extraction, between 30 and 70 mg of trimmed hair was carefully weighed, placed into a 2-mL Eppendorf tube, and 0.8-2 mL of pure methanol and 0.3-1.5 mL of milli-Q distilled water were added. Samples were incubated for up to 24 hours at 30-37° C. under moderate shaking. After incubation, extracted samples were centrifuged at 7 000-10000×g for 2 to 10 minutes, and 0.5-1 mL of supernatant was transferred into a new 2-mL Eppendorf tube. The supernatant was then placed in an oven at 38-40° C. to evaporate the extract. Once the extract was completely evaporated, the samples were reconstituted with 0.200-0.300 mL of EIA buffer and shaken for 30 seconds. Samples were then immediately stored at 20° C. until analysis.

Polyclonal or Monoclonal antibodies that specifically recognize epitopes on T3 were used as “capture antibodies.” The capture antibody was immobilized onto a solid support (e.g., ELISA plate or a cartridge). Recombinant T3 peptides or synthetic mimic peptides (e.g., 30-40 amino acids, which encompass T3 regions recognized by capture antibody) were conjugated to a detection tag, such as an enzyme (horseradish peroxidase), a fluorescent tag, radioactive tag, etc., and were used as “labeled antigens.” The sample (e.g., saliva or hair), which contains testosterone (“unlabeled antigen,”) was then mixed with the “labeled antigen,” and the mixture was brought in contact with the immobilized “capture antibody.” Both the sample (unlabeled antigen) and labeled antigen bind to the capture antibody and “compete” for binding to the limited number of capture antibodies. After the mixture was allowed to bind, the unbound sample and labeled antigen were washed off. Then a substrate (e.g., colorimetric/chromogenic substrate such as 3,3′, 5, 5′-tetramethylbenzidne (TMB) for HRP) was added. The reaction was then stopped. The enzyme-substrate reaction was detected and/or quantified using a detection platform (e.g., spectrophotometer). The signal obtained was inversely proportional to the amount of T3 (e.g., the protein of interest) in the sample.

TABLE 8
Levels of T3 detected in hair samples

| Sample | | T3 levels |
|---|---|---|
| ID | Sex | (pg/mg) |
| 1 | Female | 1.235 |
| 2 | Female | 3.405 |
| 3 | Female | 4.543 |

Table 8 shows that T3 was detected in hair. Thus, methods of the present disclosure may be applied for determining T3 levels in hair. For example, determining T3 may be useful in assessing fertility.

### Example 10: Estimating Ovarian Reserve Based on Detection of AMH in Hair

Using methods and systems of the present disclosure, Anti-Mullerian hormone (AMH) levels were measured in plasma and serum samples obtained from a plurality of subjects using an ELISA assay. Generally, ovarian AMH production may slowly decrease with increasing age of a subject until becoming undetectable in plasma or serum about five years before menopause. This ovarian AMH production level may reflect the subject's underlying ovarian reserve, as AMH serum concentration may be shown to reflect the size of the antral follicles pool, which represents the quantity of remaining primordial follicles.

As shown in Table 9, the results indicated that AMH levels measured in hair samples of a subject correlated with age of the subject more strongly than did AMH levels measured in plasma samples. These results demonstrate that measuring AMH levels from hair samples may be a superior assay approach for measuring or estimating ovarian reserve as compared to methods of assaying AMH levels from plasma samples.

TABLE 9
Average AMH levels detected in hair samples (range)

| Age | | | Number |
|---|---|---|---|
| range | | | of |
| (yrs) | Hair¹ (ng/mg) | Plasma² (ng/ml) | samples |
| <25 | 9.37 (0.32-16.3) | 3.68 (1.38-7.54) | 20 |
| 25-29 | 5.17 (0.25-16.03) | 4.6 (0.94-8.46) | 26 |
| 30-34 | 5.89 (0.34-15.28) | 3.24 (0.9-8.19) | 15 |
| 35-39 | 3.1 (0.17-13.68) | 3.34 (0.698-17.68) | 28 |
| >39 | 3.02 (0.12-13.56) | 0.92 (0-5.25) | 63 |

¹P value 1.26 × 10⁻⁵ via linear regression
²P value 0.088 via linear regression

### Example 11: Estimating Antral Follicle Count Based on Detection of AMH in Hair

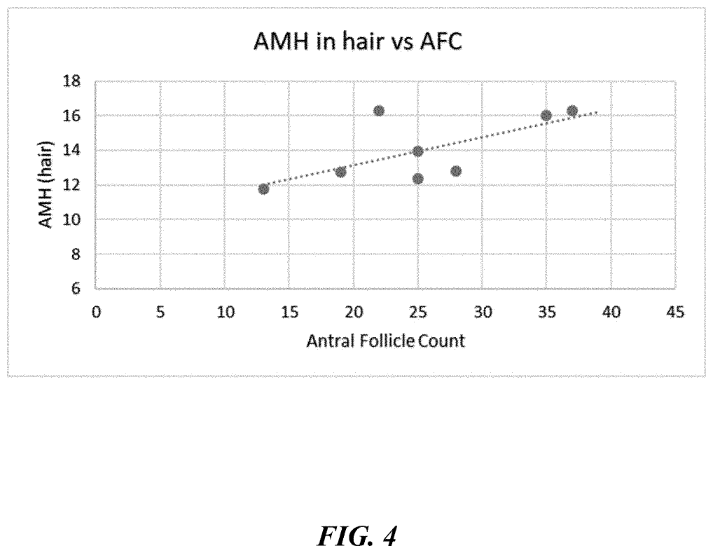

AMH levels in plasma samples may predict the size of the subject's antral follicles pool, which represents the quantity of remaining primordial follicles. Using methods and systems of the present disclosure, as shown in FIG. 4 and Table 10, AMH levels in hair samples were measured via an ELISA assay, and the results also strongly correlated with the antral follicle count (AFC) of the subject. When analysis was limited to only those hair samples with at least 150 mg of hair, AMH levels in hair samples were observed to correlate more strongly with AFC of a subject than did AMH levels in plasma (as assessed via linear regression, correcting for the size of hair samples). These results demonstrate that measuring AMH levels from hair samples may be an improved assay approach for measuring or estimating antral follicle count (AFC) as compared to methods of assaying AMH levels from plasma samples.

TABLE 10
Effect size of AMH levels in plasma and hair samples

| | | | P |
|---|---|---|---|
| | | Effect size (95% Cl) | value |
| AMH | Plasma | 1.07 (95% Cl: [−0.5, 2.6]) | 0.203 |
| | Hair | 3.75 (95% CI: [1.7, 5.8]) | 0.0168 |

### Example 12: Assay Validation for Detection of AMH in Hair

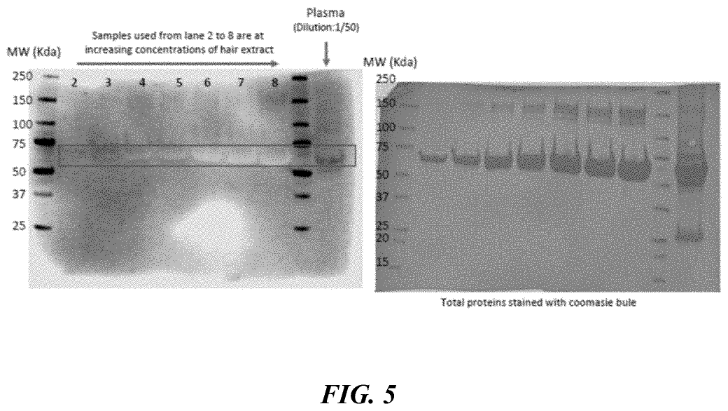

Using methods and systems of the present disclosure, an assay for detection of AMH levels in hair samples was validated. Using a Western blot, AMH levels were demonstrated to be specifically and accurately assayed in hair samples. AMH was purified from hair extract samples. When the AMH was assayed on a denaturing gel (FIG. 5, right panel), the results indicated a resolved band of about 60 kilodalton (kD), conforming to the expected molecular weight of AMH. Further, Western blotting was performed with an antibody specific for AMH, and results confirmed that this protein was indeed AMH (FIG. 5, left panel). Therefore, the assay for detection of AMH levels in hair samples was validated by these Western blotting results.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for generating a reproductive hormone profile of a subject, comprising:
(a) obtaining a hair sample of said subject;
(b) processing said hair sample of said subject to generate a data set comprising data indicative of a presence of an anti-mullerian hormone (AMH) in said hair sample; and
(c) using at least said data in (b) to generate said reproductive hormone profile of said subject.

2. The method of claim 1, wherein said subject is a human.

3. The method of claim 1, further comprising measuring a presence of another hormone selected from the group consisting of dehydroepiandrosterone, estradiol, progesterone, testosterone, and cortisol.

4. The method of claim 1, wherein said reproductive hormone profile is an assessment of ovarian reserve in said subject.

5. The method of claim 1, wherein said reproductive hormone profile is an assessment of reproductive lifespan of said subject.

6. The method of claim 1, wherein said reproductive hormone profile is an assessment of ovarian dysfunction in said subject.

7. The method of claim 6, wherein said ovarian dysfunction is selected from the group consisting of polycystic ovary syndrome, endometriosis, anovulation, persistent follicles, and granulosa cell cancer.

8. The method of claim 1, wherein said reproductive hormone profile is an assessment of said subject after said subject undergoes gonadotoxic cancer treatment.

9. The method of claim 1, wherein said reproductive hormone profile is an assessment of said subject after said subject undergoes a complete oophorectomy.

10. The method of claim 1, wherein said reproductive hormone profile is an assessment of said subject after said subject undergoes a partial oophorectomy.

11. A system for generating a reproductive hormone profile of a subject, comprising:
a database comprising reference values of an anti-mullerian hormone (AMH);
a communications interface; and
a computer processer operatively coupled to said database and said communications interface, wherein said computer processor is programmed to (i) receive a request to process a hair sample of said subject to generate data indicative of a presence of said AMH in said hair sample; (ii) generate an output, which output comprises said reproductive hormone profile of said subject based on at least said data of (i) to said reference values of said AMH in said database; and (iii) display said output on said communications interface.

12. The system of claim 11, wherein said computer processor is further programmed to measure said presence of another hormone selected from the group consisting of dehydroepiandrosterone, estradiol, progesterone, testosterone, and cortisol.

13. The system of claim 11, wherein said reproductive hormone profile is an assessment of ovarian reserve in said subject.

14. The system of claim 11, wherein said reproductive hormone profile is an assessment of reproductive lifespan of said subject.

15. The system of claim 11, wherein said reproductive hormone profile is an assessment of ovarian dysfunction in said subject.

16. The system of claim 15, wherein said ovarian dysfunction is selected from the group consisting of polycystic ovary syndrome, endometriosis, anovulation, persistent follicles, and granulosa cell cancer.

17. The system of claim 11, wherein said reproductive hormone profile is an assessment of said subject after said subject undergoes gonadotoxic cancer treatment.

18. The system of claim 11, wherein said reproductive hormone profile is an assessment of said subject after said subject undergoes a complete oophorectomy.

19. The system of claim 11, wherein said reproductive hormone profile is an assessment of said subject after said subject undergoes a partial oophorectomy.

20. A method of identifying or quantifying anti-mullerian hormone (AMH) in a hair sample of a subject, said method comprising:
a) obtaining said hair sample of said subject;
b) processing said hair sample to produce a processed sample; and
c) identifying or quantifying said anti-mullerian hormone in said processed sample from b).
